(19) United States
(12) Patent Application Publication
(10) Pub. No.: US 2021/0093824 A1
(43) Pub. Date: Apr. 1, 2021

(54) METHODS AND APPARATUS FOR TREATING A RESPIRATORY DISORDER

(71) Applicant: ResMed Pty Ltd, Bella Vista, NSW (AU)

(72) Inventor: Michael Waclaw COLEFAX, Sydney (AU)

(73) Assignee: ResMed Pty Ltd, Bella Vista, NSW (AU)

(21) Appl. No.: 17/042,609

(22) PCT Filed: Apr. 5, 2019
(86) PCT No.: PCT/AU2019/050302

§ 371 Date: Sep. 28, 2020

(30) Foreign Application Priority Data
Apr. 6, 2018 (AU) 2018901147

(51) Int. Cl. A61M 16/10; A61M 16/00; A61M 16/20; B01D 53/04; B01D 53/047

(52) U.S. Cl. CPC A61M 16/101; B01D 2259/4541; A61M 16/208; B01D 53/0415; B01D 53/047; A61M 2016/0027; A61M 2230/42; A61M 2205/3372; B01D 2253/108; B01D 2256/12; B01D 2257/102; B01D 2259/40009; B01D 2259/402; B01D 2259/4533; A61M 16/024

(57) ABSTRACT

Method(s) and apparatus provide a controlled release of enriched gas such as the gas produced by an oxygen concentrator (100) using adaptive triggering. Release of a bolus may be responsive to a generated trigger signal. The trigger signal may be generated by an evaluation of a trigger threshold. The trigger threshold may be derived from or calculated from a pressure signal, such as an adjusted pressure signal, from a pressure sensor. The pressure sensor may be pneumatically coupled with an airway of a user such that the pressure signal may be representative of airway pressure, or changes in airway pressure, attributable to the user. The trigger signal may be generated from a comparison between the pressure signal and the trigger threshold. The trigger threshold may be derived with an activity signal, such as one computed from the pressure signal, so as to adapt trigging sensitivity.

### CROSS-REFERENCE TO RELATED APPLICATIONS

This application claims the benefit of Australian Provisional Application No. 2018901147, filed 6 Apr. 2018, the entire disclosure of which is hereby incorporated herein by reference.

### FIELD OF THE TECHNOLOGY

The present technology relates generally to methods and apparatus for treating respiratory disorders, such as to control operation(s) of an oxygen concentrator (e.g., a portable oxygen concentrator), such as for increasing the efficiency of a pulsed oxygen delivery.

### DESCRIPTION OF THE RELATED ART

There are many users that require supplemental oxygen as part of Long Term Oxygen Therapy (LTOT). Currently, the vast majority of users that are receiving LTOT are diagnosed under the general category of Chronic Obstructive Pulmonary Disease (COPD). This general diagnosis includes such common diseases as Chronic Asthma, Emphysema, and several other cardio-pulmonary conditions. Other users may also require supplemental oxygen, for example, obese individuals to maintain elevated activity levels, or infants with cystic fibrosis or broncho-pulmonary dysplasia.

Doctors may prescribe oxygen concentrators or portable tanks of medical oxygen for these users. Usually a specific continuous oxygen flow rate is prescribed (e.g., 1 litre per minute (LPM), 2 LPM, 3 LPM, etc.). Experts in this field have also recognized that exercise for these users provide long term benefits that slow the progression of the disease, improve quality of life and extend user longevity. Most stationary forms of exercise like tread mills and stationary bicycles, however, are too strenuous for these users. As a result, the need for mobility has long been recognized. Until recently, this mobility has been facilitated by the use of small compressed oxygen tanks. The disadvantage of these tanks is that they have a finite amount of oxygen and they are heavy, weighing about 50 pounds, when mounted on a cart with dolly wheels.

Oxygen concentrators have been in use for about 50 years to supply users suffering from respiratory insufficiency with supplemental oxygen. Traditional oxygen concentrators used to provide these flow rates have been bulky and heavy making ordinary ambulatory activities with them difficult and impractical. Recently, companies that manufacture large stationary home oxygen concentrators began developing portable oxygen concentrators (POCs). The advantage of POCs is that they can produce a theoretically endless supply of oxygen. In order to make these devices small for mobility, the various systems necessary for the production of oxygen enriched gas are condensed.

Portable oxygen concentrators seek to utilize their produced oxygen as efficiently as possible, in order to minimise weight, size, and power consumption. This may be achieved by delivering oxygen as a pulse or bolus timed to coincide with the onset of inhalation, in a mode known as pulsed or demand oxygen delivery (POD). POD is more efficient than continuous flow delivery, since oxygen delivered during exhalation is wasted.

In POD mode therapy, the onset of each inhalation is detected to trigger the release of an oxygen bolus. Typically, this is done by analysis of a pressure signal generated by a pressure sensor in fluid communication with the oxygen delivery conduit to detect a sudden fall in pressure, since at inhalation onset the conduit pressure drops below ambient. An algorithm for this purpose is required to operate in real time with as low trigger latency as possible, and to function reliably with varying levels of signal strength and signal to noise ratio.

Conventional POD triggering algorithms are based on the comparison of the pressure signal with a trigger threshold. The trigger threshold may be fixed, or manually selectable from multiple predetermined values. The predetermined trigger threshold values are typically sensitive, of the order of −1 mmH₂O. Greater sensitivity, and conversely lower immunity to noise, correlates with a lower trigger threshold magnitude.

However, during user activity (e.g. walking), additional noise is captured on the pressure signal that may increase the rate of false triggers, i.e. triggers that do not coincide with onset of inhalation. This may also occur in the presence of other external noise or vibration such as while the POC is being used in a moving vehicle, placed on a trolley being rolled over a rough surface, or if the conduit is periodically bumped or shaken. False triggers lead to wasted oxygen and hence lower efficiency. In addition, during long inhalations, conventional algorithms may trigger twice. The second trigger is usually so late in inhalation that the second delivered bolus is wasted.

Conversely, in some scenarios the pressure signal becomes too weak for the fixed threshold(s) to reliably detect onset of inhalation. These scenarios may include (a) during low minute volume respiration, e.g. small-sized user during sleep, (b) mouth breathing during sleep, and (c) a displaced cannula. In these scenarios the needed bolus is not delivered, compromising the therapy effectiveness.

There is therefore a need to improve triggering of POD therapy.

### SUMMARY OF THE TECHNOLOGY

Example methods and apparatus of the present technology may involve control of a therapy for a respiratory disorder. The technology may involve control of release of pulsed oxygen. In some examples, a threshold may be implemented for control of the release of the pulsed oxygen, such as by generating a control signal to activate a valve. The threshold for triggering may be adaptive, being repeatedly calculated based on the characteristics of a signal representing a user's airway pressure, such that the triggering becomes more sensitive as the patient's activity level decreases, and/or vice versa (i.e., less sensitive as the patient's activity level increases). The disclosed method may optionally also monitor each breath after a trigger signal is generated, and not allow a subsequent trigger signal to be generated until exhalation has occurred.

Some versions of the present technology may include a method of generating a trigger signal for controlling release of a bolus of oxygen enriched gas from an oxygen concentrator. The method may include calculating a trigger threshold from a pressure signal representing an airway pressure of a user. The method may include comparing the pressure signal with the trigger threshold. The method may include generating, based on the comparison, the trigger signal for controlling release of the bolus.

In some versions, calculating the trigger threshold may include computing an activity signal from the pressure signal. The activity signal may represent activity other than respiration activity. Calculating the trigger threshold may include decreasing a sensitivity of the trigger threshold with an increase in an indication of activity in the activity signal. Decreasing the sensitivity of the trigger threshold may include making the trigger threshold more negative. Calculating the trigger threshold may include increasing a sensitivity of the trigger threshold with a decrease in an indication of activity in the activity signal. Increasing the sensitivity of the trigger threshold may include making the trigger threshold less negative.

In some versions, the indication of activity may be derived as a function of a window of values of the activity signal. A duration of the window may vary as a function of time since the trigger threshold exceeded an average of trigger threshold values. The function of time may be configured to shorten the duration of the window. The function of time may be further configured to gradually increase the duration of the window to a limit.

In some versions, calculating the trigger threshold may include setting the trigger threshold according to a function of (a) a scaling constant, and (b) a maximum value of a window of values of the activity signal. The function may include multiplying the scaling constant and the maximum value and reversing a sign of a value of the function. The method may further include varying the scaling constant with the maximum value. The method may further include varying the scaling constant with a breathing rate of the user. Computing the activity signal may include high pass filtering the pressure signal. Comparing the pressure signal with the trigger threshold may include determining whether the pressure signal falls below the trigger threshold continuously for at least a trigger confirmation period. Generating the trigger signal may include asserting a Boolean trigger signal. The method may further include detecting an expiration of the user. Assertion of the Boolean trigger signal may be conditioned on a detection of an expiration since the last assertion of the Boolean trigger signal. Detecting an expiration may include determining whether the pressure signal remains above an expiratory threshold for a minimum expiration period. Assertion of the Boolean trigger signal may be conditioned on a time since the last assertion of the Boolean trigger signal exceeding a minimum time between boluses. Assertion of the Boolean trigger signal may be conditioned on a duration of a current inspiration being greater than a minimum inspiratory time. The method may further include calculating the minimum inspiratory time as a function of a recent average inspiratory time. Calculating the minimum inspiratory time may include choosing a value between a floor value and a ceiling value. At least one of the floor value and the ceiling value may increase with the recent average inspiratory time.

In some versions, the pressure signal may be an adjusted pressure signal. The method may further include generating the adjusted pressure signal by computing values for the adjusted pressure signal that adjust at least one period of a measured pressure signal that coincides with a bolus release to remove an effect of the bolus release on the measured pressure signal. Computing values for the adjusted pressure signal may include interpolating between a last measured pressure value prior to the bolus release and a first measured pressure value after the bolus release. Computing values for the adjusted pressure signal may further include interpolating values for a settling period of the adjusted pressure signal that may occur after the first measured pressure value. The adjusted pressure signal may be generated by filtering so as to achieve one or both of: (a) removal of very short duration, large magnitude impulses, and (b) removal of periodic device noise. The adjusted pressure signal may be generated by compensating for a temperature of the oxygen concentrator. Compensating for the temperature may include computing a pressure offset from a signal representing the temperature of the oxygen concentrator. The method may further include estimating a breathing rate of the user from successive instants of bolus release. The method may further include estimating an inspiratory time of the user.

Some versions of the present technology may include a computer-readable medium having encoded thereon computer-readable instructions that when executed by a controller of an oxygen concentrator cause the controller to perform any of the methods described herein, such as including any of the aspects of the adaptive triggering methods described herein.

Some versions of the present technology may include a portable oxygen concentrator. The portable oxygen concentrator may include an outlet. The outlet may be suitable for pneumatic coupling with a delivery device. The delivery device may be configured for delivering, in use, oxygen enriched gas to a user. The portable oxygen concentrator may include one or more, or at least two, canisters including gas separation adsorbent. The gas separation adsorbent may be configured for gas separation of at least some nitrogen from air in the canister(s) to produce the oxygen enriched gas. The portable oxygen concentrator may include a compression system that may include a compressor coupled to at least one of the canisters to compress air during operation to promote the gas separation. The portable oxygen concentrator may include an accumulator coupled to one or more of the canisters, to accumulate the oxygen enriched gas produced in one or more of the canisters during use. The accumulator may be pneumatically coupled to the outlet. The portable oxygen concentrator may include one or more sensors. The portable oxygen concentrator may include a controller, such as with one or more processors, and a set of valves that are coupled to the controller. The controller may be configured to control operation of the set of valves to produce the oxygen enriched gas into the accumulator. The controller may be configured to control release of the produced oxygen enriched gas from the accumulator in at least one bolus. The controller may be further configured to operate with any of the computer-readable medium(s) described herein, such as including any of the aspects of the adaptive triggering methods described herein.

Some versions of the present technology may include an adaptive triggering system for an oxygen concentrator. The system may include a threshold module configured to repeatedly calculate a trigger threshold from a pressure signal representing an airway pressure of a user. The system may include a trigger module. The trigger module may be configured to compare the pressure signal with the trigger threshold. The trigger module may be configured to generate, based on the comparison, a trigger signal to control release of a bolus.

In some versions of the adaptive triggering system, the pressure signal may be an adjusted pressure signal. The system may further include a pressure module configured to generate the adjusted pressure signal by adjusting at least one period of a measured pressure signal that coincides with a bolus release to remove an effect of the bolus release on the measured pressure signal. The system may further include a temperature sensor configured to generate a temperature signal. The pressure module may be configured to generate the adjusted pressure signal using the temperature signal to compensate for temperature of the oxygen concentrator. The system may further include a monitoring module configured to calculate one or more breathing parameters of the user from successive instants of bolus release.

Some versions of the present technology may include an oxygen concentrator that may include the adaptive triggering system as described herein.

Some versions of the present technology may include an adaptive triggering system for an oxygen concentrator. The system may include means for repeatedly calculating a trigger threshold from a pressure signal representing an airway pressure of a user. The system may include means for comparing the pressure signal with the trigger threshold. The system may include means for generating a trigger signal to control release of a bolus of oxygen based on the comparison.

### BRIEF DESCRIPTION OF THE DRAWINGS

Advantages of the present technology will become apparent to those skilled in the art with the benefit of the following detailed description of embodiments and upon reference to the accompanying drawings in which:

FIG. 1 depicts a schematic diagram of the components of an oxygen concentrator;

FIG. 2 depicts a side view of the main components of an oxygen concentrator;

FIG. 3A depicts a perspective side view of a compression system;

FIG. 3B depicts a side view of a compression system that includes a heat exchange conduit;

FIG. 4A depicts a schematic diagram of example outlet components of an oxygen concentrator;

FIG. 4B depicts an outlet conduit for an oxygen concentrator;

FIG. 4C depicts an alternate outlet conduit for an oxygen concentrator;

FIG. 5 depicts an outer housing for an oxygen concentrator;

FIG. 6 depicts an example control panel for an oxygen concentrator;

FIG. 7 is a block diagram illustrating a components or modules and signals of a system configured to trigger release of a bolus of oxygen from an oxygen concentrator such as the one of FIG. 1 according to one implementation of the present technology.

FIG. 8 is a block diagram illustrating one implementation of the trigger module of the system of FIG. 7 according to the present technology.

FIG. 9 is a block diagram illustrating one implementation of the threshold module of the system of FIG. 7 according to the present technology.

FIG. 10 is a block diagram illustrating one implementation of the trigger module of the system of FIG. 7 according to the present technology.

FIG. 11 is a graph illustrating the value of a scaling constant as a function of the maximum activity according to one implementation of the present technology.

FIG. 12 is a graph illustrating another implementation of window duration adjustment.

While the present technology is susceptible to various modifications and alternative forms, specific embodiments thereof are shown by way of example in the drawings and will herein be described in detail. It should be understood, however, that the drawings and detailed description thereto are not intended to limit the technology to the particular form disclosed, but on the contrary, the intention is to cover all modifications, equivalents, and alternatives falling within the spirit and scope of the present technology as defined by the appended claims.

### DETAILED DESCRIPTION OF THE EMBODIMENTS

It is to be understood the present technology is not limited to particular devices or methods, which may, of course, vary. It is also to be understood that the terminology used herein is for the purpose of describing particular embodiments only, and is not intended to be limiting. Headings are for organizational purposes only and are not meant to be used to limit or interpret the description or claims. As used in this specification and the appended claims, the singular forms “a”, “an”, and “the” include singular and plural referents unless the content clearly dictates otherwise. Furthermore, the word “may” is used throughout this application in a permissive sense (i.e., having the potential to, being able to), not in a mandatory sense (i.e., must). The term “include,” and derivations thereof, mean “including, but not limited to.”

The term “coupled” as used herein means either a direct connection or an indirect connection (e.g., one or more intervening connections) between one or more objects or components. The phrase “connected” means a direct connection between objects or components such that the objects or components are connected directly to each other. As used herein the phrase “obtaining” a device means that the device is either purchased or constructed.

Oxygen concentrators take advantage of pressure swing adsorption (PSA). Pressure swing adsorption may involve using a compressor to increase gas pressure inside a canister that contains particles of a gas separation adsorbent. As the pressure increases, certain molecules in the gas may become adsorbed onto the gas separation adsorbent. Removal of a portion of the gas in the canister under the pressurized conditions allows separation of the non-adsorbed molecules from the adsorbed molecules. The gas separation adsorbent may be regenerated by reducing the pressure, which reverses the adsorption of molecules from the adsorbent. Further details regarding oxygen concentrators may be found, for example, in U.S. Published Patent Application No. 2009-0065007, published Mar. 12, 2009, and entitled “Oxygen Concentrator Apparatus and Method”, which is incorporated herein by reference.

Ambient air usually includes approximately 78% nitrogen and 21% oxygen with the balance comprised of argon, carbon dioxide, water vapor and other trace gases. If a gas mixture such as air, for example, is passed under pressure through a vessel containing a gas separation adsorbent bed that attracts nitrogen more strongly than it does oxygen, part or all of the nitrogen will stay in the bed, and the gas coming out of the vessel will be enriched in oxygen. When the bed reaches the end of its capacity to adsorb nitrogen, it can be regenerated by reducing the pressure, thereby releasing the adsorbed nitrogen. It is then ready for another cycle of producing oxygen enriched air. By alternating canisters in a two-canister system, one canister can be collecting oxygen while the other canister is being purged (resulting in a continuous separation of the oxygen from the nitrogen). In this manner, oxygen can be accumulated out of the air for a variety of uses include providing supplemental oxygen to users.

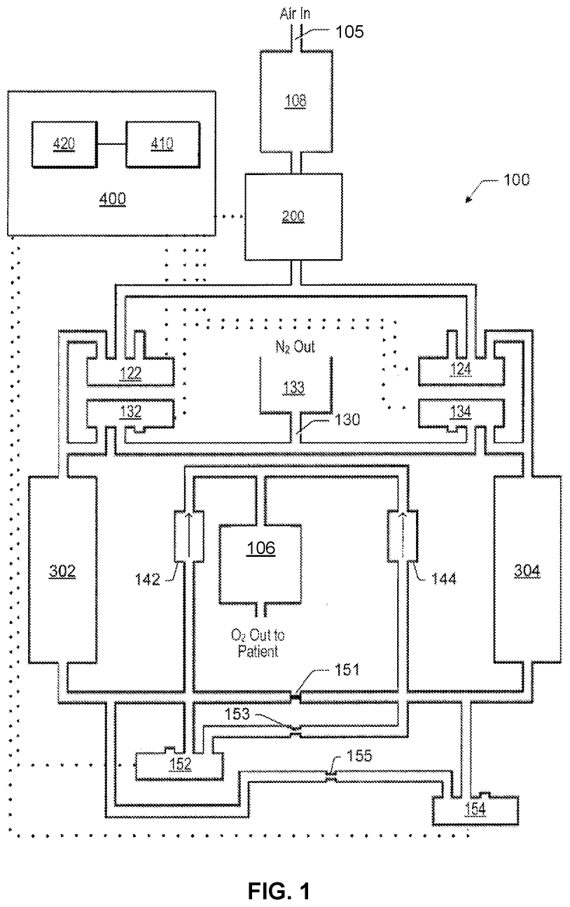

FIG. 1 illustrates a schematic diagram of an oxygen concentrator 100, according to an implementation. Oxygen concentrator 100 may concentrate oxygen out of an air stream to provide oxygen enriched gas to a user. As used herein, “oxygen enriched gas” is composed of at least about 50% oxygen, at least about 60% oxygen, at least about 70% oxygen, at least about 80% oxygen, at least about 90% oxygen, at least about 95% oxygen, at least about 98% oxygen, or at least about 99% oxygen.

Oxygen concentrator 100 may be a portable oxygen concentrator. For example, oxygen concentrator 100 may have a weight and size that allows the oxygen concentrator to be carried by hand and/or in a carrying case. In one implementation, oxygen concentrator 100 has a weight of less than about 20 lbs., less than about 15 lbs., less than about 10 lbs, or less than about 5 lbs. In an implementation, oxygen concentrator 100 has a volume of less than about 1000 cubic inches, less than about 750 cubic inches; less than about 500 cubic inches, less than about 250 cubic inches, or less than about 200 cubic inches.

Oxygen may be collected from ambient air by pressurising ambient air in canisters 302 and 304, which include a gas separation adsorbent. Gas separation adsorbents useful in an oxygen concentrator are capable of separating at least nitrogen from an air stream to produce oxygen enriched gas. Examples of gas separation adsorbents include molecular sieves that are capable of separation of nitrogen from an air stream. Examples of adsorbents that may be used in an oxygen concentrator include, but are not limited to, zeolites (natural) or synthetic crystalline aluminosilicates that separate nitrogen from oxygen in an air stream under elevated pressure. Examples of synthetic crystalline aluminosilicates that may be used include, but are not limited to: OXYSIV adsorbents available from UOP LLC, Des Plaines, Iowa; SYLOBEAD adsorbents available from W. R. Grace & Co, Columbia, Md.; SILIPORITE adsorbents available from CECA S. A. of Paris, France; ZEOCHEM adsorbents available from Zeochem AG, Uetikon, Switzerland; and AgLiLSX adsorbent available from Air Products and Chemicals, Inc., Allentown, Pa.

As shown in FIG. 1, air may enter the oxygen concentrator through air inlet 105. Air may be drawn into air inlet 105 by compression system 200. Compression system 200 may draw in air from the surroundings of the oxygen concentrator and compress the air, forcing the compressed air into one or both canisters 302 and 304. In an implementation, an inlet muffler 108 may be coupled to air inlet 105 to reduce sound produced by air being pulled into the oxygen concentrator by compression system 200. In an implementation, inlet muffler 108 may be a moisture and sound absorbing muffler. For example, a water absorbent material (such as a polymer water absorbent material or a zeolite material) may be used to both absorb water from the incoming air and to reduce the sound of the air passing into the air inlet 105.

Compression system 200 may include one or more compressors capable of compressing air. Pressurized air, produced by compression system 200, may be forced into one or both of the canisters 302 and 304. In some implementations, the ambient air may be pressurized in the canisters to a pressure approximately in a range of 13-20 pounds per square inch (psi). Other pressures may also be used, depending on the type of gas separation adsorbent disposed in the canisters.

Coupled to each canister 302/304 are inlet valves 122/124 and outlet valves 132/134. As shown in FIG. 1, inlet valve 122 is coupled to canister 302 and inlet valve 124 is coupled to canister 304. Outlet valve 132 is coupled to canister 302 and outlet valve 134 is coupled to canister 304. Inlet valves 122/124 are used to control the passage of air from compression system 200 to the respective canisters. Outlet valves 132/134 are used to release gas from the respective canisters during a venting process. In some implementations, inlet valves 122/124 and outlet valves 132/134 may be silicon plunger solenoid valves. Other types of valves, however, may be used. Plunger valves offer advantages over other kinds of valves by being quiet and having low slippage.

In some implementations, a two-step valve actuation voltage may be used to control inlet valves 122/124 and outlet valves 132/134. For example, a high voltage (e.g., 24 V) may be applied to an inlet valve to open the inlet valve. The voltage may then be reduced (e.g., to 7 V) to keep the inlet valve open. Using less voltage to keep a valve open may use less power (Power=Voltage*Current). This reduction in voltage minimizes heat build up and power consumption to extend run time from the battery. When the power is cut off to the valve, it closes by spring action. In some implementations, the voltage may be applied as a function of time that is not necessarily a stepped response (e.g., a curved downward voltage between an initial 24 V and a final 7 V).

In an implementation, pressurized air is sent into one of canisters 302 or 304 while the other canister is being vented. For example, during use, inlet valve 122 is opened while inlet valve 124 is closed. Pressurized air from compression system 200 is forced into canister 302, while being inhibited from entering canister 304 by inlet valve 124. In an implementation, a controller 400 is electrically coupled to valves 122, 124, 132, and 134. Controller 400 includes one or more processors 410 operable to execute program instructions stored in memory 420. The program instructions are operable to perform various predefined methods that are used to operate the oxygen concentrator, such as the methods described in more detail herein. Controller 400 may include program instructions for operating inlet valves 122 and 124 out of phase with each other, i.e., when one of inlet valves 122 or 124 is opened, the other valve is closed. During pressurization of canister 302, outlet valve 132 is closed and outlet valve 134 is opened. Similar to the inlet valves, outlet valves 132 and 134 are operated out of phase with each other. In some implementations, the voltages and the duration of the voltages used to open the input and output valves may be controlled by controller 400.

Check valves 142 and 144 are coupled to canisters 302 and 304, respectively. Check valves 142 and 144 are one-way valves that are passively operated by the pressure differentials that occur as the canisters are pressurized and vented. Check valves 142 and 144 are coupled to canisters to allow oxygen produced during pressurization of the canister to flow out of the canister, and to inhibit back flow of oxygen or any other gases into the canister. In this manner, check valves 142 and 144 act as one-way valves allowing oxygen enriched gas to exit the respective canister during pressurization.

The term “check valve”, as used herein, refers to a valve that allows flow of a fluid (gas or liquid) in one direction and inhibits back flow of the fluid. Examples of check valves that are suitable for use include, but are not limited to: a ball check valve; a diaphragm check valve; a butterfly check valve; a swing check valve; a duckbill valve; and a lift check valve. Under pressure, nitrogen molecules in the pressurized ambient air are adsorbed by the gas separation adsorbent in the pressurized canister. As the pressure increases, more nitrogen is adsorbed until the gas in the canister is enriched in oxygen. The nonadsorbed gas molecules (mainly oxygen) flow out of the pressurized canister when the pressure reaches a point sufficient to overcome the resistance of the check valve coupled to the canister. In one implementation, the pressure drop of the check valve in the forward direction is less than 1 psi. The break pressure in the reverse direction is greater than 100 psi. It should be understood, however, that modification of one or more components would alter the operating parameters of these valves. If the forward flow pressure is increased, there is, generally, a reduction in oxygen enriched gas production. If the break pressure for reverse flow is reduced or set too low, there is, generally, a reduction in oxygen enriched gas pressure.

In an exemplary implementation, canister 302 is pressurized by compressed air produced in compression system 200 and passed into canister 302. During pressurization of canister 302 inlet valve 122 is open, outlet valve 132 is closed, inlet valve 124 is closed and outlet valve 134 is open. Outlet valve 134 is opened when outlet valve 132 is closed to allow substantially simultaneous venting of canister 304 while canister 302 is pressurized. Canister 302 is pressurized until the pressure in canister is sufficient to open check valve 142. Oxygen enriched gas produced in canister 302 exits through check valve and, in one implementation, is collected in accumulator 106.

After some time, the gas separation adsorbent will become saturated with nitrogen and will be unable to separate significant amounts of nitrogen from incoming air. This point is usually reached after a predetermined time of oxygen enriched gas production. In the implementation described above, when the gas separation adsorbent in canister 302 reaches this saturation point, the inflow of compressed air is stopped and canister 302 is vented to remove nitrogen. During venting, inlet valve 122 is closed, and outlet valve 132 is opened. While canister 302 is being vented, canister 304 is pressurized to produce oxygen enriched gas in the same manner described above. Pressurization of canister 304 is achieved by closing outlet valve 134 and opening inlet valve 124. The oxygen enriched gas exits canister 304 through check valve 144.

During venting of canister 302, outlet valve 132 is opened allowing pressurized gas (mainly nitrogen) to exit the canister through concentrator outlet 130. In an implementation, the vented gases may be directed through muffler 133 to reduce the noise produced by releasing the pressurized gas from the canister. As gas is released from canister 302, the pressure in the canister drops, allowing the nitrogen to become desorbed from the gas separation adsorbent. The released nitrogen exits the canister through outlet 130, resetting the canister to a state that allows renewed separation of oxygen from an air stream. Muffler 133 may include open cell foam (or another material) to muffle the sound of the gas leaving the oxygen concentrator. In some implementations, the combined muffling components/techniques for the input of air and the output of gas, may provide for oxygen concentrator operation at a sound level below 50 decibels.

During venting of the canisters, it is advantageous that at least a majority of the nitrogen is removed. In an implementation, at least about 50%, at least about 60%, at least about 70%, at least about 80%, at least about 90%, at least about 95%, at least about 98%, or substantially all of the nitrogen in a canister is removed before the canister is re-used to separate oxygen from air. In some implementations, a canister may be further purged of nitrogen using an oxygen enriched stream that is introduced into the canister from the other canister.

In an exemplary implementation, a portion of the oxygen enriched gas may be transferred from canister 302 to canister 304 when canister 304 is being vented of nitrogen. Transfer of oxygen enriched gas from canister 302 to 304, during venting of canister 304, helps to further purge nitrogen (and other gases) from the canister. In an implementation, oxygen enriched gas may travel through flow restrictors 151, 153, and 155 between the two canisters. Flow restrictor 151 may be a trickle flow restrictor. Flow restrictor 151, for example, may be a 0.009D flow restrictor (e.g., the flow restrictor has a radius 0.009″ which is less than the diameter of the tube it is inside). Flow restrictors 153 and 155 may be 0.013D flow restrictors. Other flow restrictor types and sizes are also contemplated and may be used depending on the specific configuration and tubing used to couple the canisters. In some implementations, the flow restrictors may be press fit flow restrictors that restrict air flow by introducing a narrower diameter in their respective tube. In some implementations, the press fit flow restrictors may be made of sapphire, metal or plastic (other materials are also contemplated).

Flow of oxygen enriched gas is also controlled by use of valve 152 and valve 154. Valves 152 and 154 may be opened for a short duration during the venting process (and may be closed otherwise) to prevent excessive oxygen loss out of the purging canister. Other durations are also contemplated. In an exemplary implementation, canister 302 is being vented and it is desirable to purge canister 302 by passing a portion of the oxygen enriched gas being produced in canister 304 into canister 302. A portion of oxygen enriched gas, upon pressurization of canister 304, will pass through flow restrictor 151 into canister 302 during venting of canister 302. Additional oxygen enriched air is passed into canister 302, from canister 304, through valve 154 and flow restrictor 155. Valve 152 may remain closed during the transfer process, or may be opened if additional oxygen enriched gas is needed. The selection of appropriate flow restrictors 151 and 155, coupled with controlled opening of valve 154 allows a controlled amount of oxygen enriched gas to be sent from canister 304 to 302. In an implementation, the controlled amount of oxygen enriched gas is an amount sufficient to purge canister 302 and minimize the loss of oxygen enriched gas through venting valve 132 of canister 302. While this implementation describes venting of canister 302, it should be understood that the same process can be used to vent canister 304 using flow restrictor 151, valve 152 and flow restrictor 153.

The pair of equalization/vent valves 152/154 work with flow restrictors 153 and 155 to optimize the air flow balance between the two canisters. This may allow for better flow control for venting the canisters with oxygen enriched gas from the other of the canisters. It may also provide better flow direction between the two canisters. It has been found that, while flow valves 152/154 may be operated as bi-directional valves, the flow rate through such valves varies depending on the direction of fluid flowing through the valve. For example, oxygen enriched gas flowing from canister 304 toward canister 302 has a flow rate faster through valve 152 than the flow rate of oxygen enriched gas flowing from canister 302 toward canister 304 through valve 152. If a single valve was to be used, eventually either too much or too little oxygen enriched gas would be sent between the canisters and the canisters would, over time, begin to produce different amounts of oxygen enriched gas. Use of opposing valves and flow restrictors on parallel air pathways may equalize the flow pattern of the oxygen between the two canisters. Equalising the flow may allow for a steady amount of oxygen available to the user over multiple cycles and also may allow a predictable volume of oxygen to purge the other of the canisters. In some implementations, the air pathway may not have restrictors but may instead have a valve with a built-in resistance or the air pathway itself may have a narrow radius to provide resistance.

At times, oxygen concentrator may be shut down for a period of time. When an oxygen concentrator is shut down, the temperature inside the canisters may drop as a result of the loss of adiabatic heat from the compression system. As the temperature drops, the volume occupied by the gases inside the canisters will drop. Cooling of the canisters may lead to a negative pressure in the canisters. Valves (e.g., valves 122, 124, 132, and 134) leading to and from the canisters are dynamically sealed rather than hermetically sealed. Thus, outside air may enter the canisters after shutdown to accommodate the pressure differential. When outside air enters the canisters, moisture from the outside air may condense inside the canister as the air cools. Condensation of water inside the canisters may lead to gradual degradation of the gas separation adsorbents, steadily reducing ability of the gas separation adsorbents to produce oxygen enriched gas.

In an implementation, outside air may be inhibited from entering canisters after the oxygen concentrator is shutdown by pressurising both canisters prior to shutdown. By storing the canisters under a positive pressure, the valves may be forced into a hermetically closed position by the internal pressure of the air in the canisters. In an implementation, the pressure in the canisters, at shutdown, should be at least greater than ambient pressure. As used herein the term “ambient pressure” refers to the pressure of the surroundings in which the oxygen concentrator is located (e.g. the pressure inside a room, outside, in a plane, etc.). In an implementation, the pressure in the canisters, at shutdown, is at least greater than standard atmospheric pressure (i.e., greater than 760 mmHg (Torr), 1 atm, 101,325 Pa). In an implementation, the pressure in the canisters, at shutdown, is at least about 1.1 times greater than ambient pressure; is at least about 1.5 times greater than ambient pressure; or is at least about 2 times greater than ambient pressure.

In an implementation, pressurization of the canisters may be achieved by directing pressurized air into each canister from the compression system and closing all valves to trap the pressurized air in the canisters. In an exemplary implementation, when a shutdown sequence is initiated, inlet valves 122 and 124 are opened and outlet valves 132 and 134 are closed. Because inlet valves 122 and 124 are joined together by a common conduit, both canisters 302 and 304 may become pressurized as air and or oxygen enriched gas from one canister may be transferred to the other canister. This situation may occur when the pathway between the compression system and the two inlet valves allows such transfer. Because the oxygen concentrator operates in an alternating pressurize/venting mode, at least one of the canisters should be in a pressurized state at any given time. In an alternate implementation, the pressure may be increased in each canister by operation of compression system 200. When inlet valves 122 and 124 are opened, pressure between canisters 302 and 304 will equalize, however, the equalized pressure in either canister may not be sufficient to inhibit air from entering the canisters during shutdown. In order to ensure that air is inhibited from entering the canisters, compression system 200 may be operated for a time sufficient to increase the pressure inside both canisters to a level at least greater than ambient pressure. Regardless of the method of pressurization of the canisters, once the canisters are pressurized, inlet valves 122 and 124 are closed, trapping the pressurized air inside the canisters, which inhibits air from entering the canisters during the shutdown period.

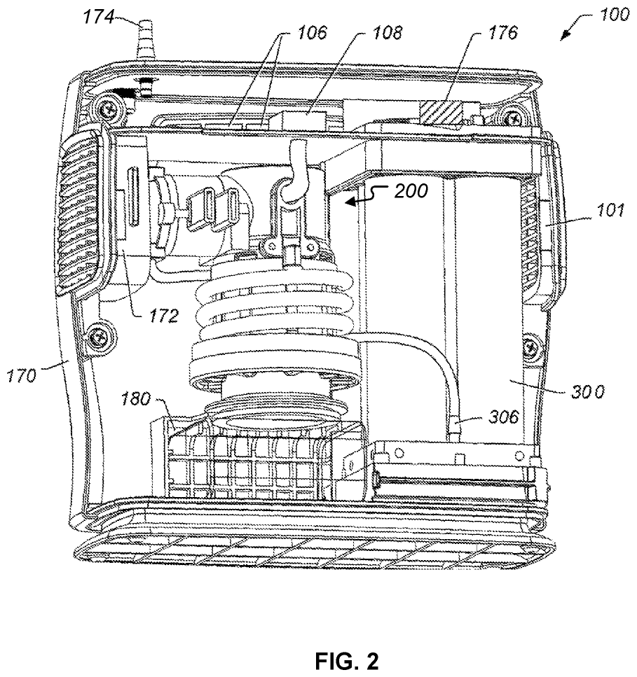

Referring to FIG. 2, an implementation of an oxygen concentrator 100 is depicted. Oxygen concentrator 100 includes a compression system 200, a canister assembly 300, and a power supply 180 disposed within an outer housing 170. Inlets 101 are located in outer housing 170 to allow air from the environment to enter oxygen concentrator 100. Inlets 101 may allow air to flow into the compartment to assist with cooling of the components in the compartment. Power supply 180 provides a source of power for the oxygen concentrator 100. Compression system 200 draws air in through the inlet 105 and muffler 108. Muffler 108 may reduce noise of air being drawn in by the compression system and also may include a desiccant material to remove water from the incoming air. Oxygen concentrator 100 may further include fan 172 used to vent air and other gases from the oxygen concentrator.

### Compression System

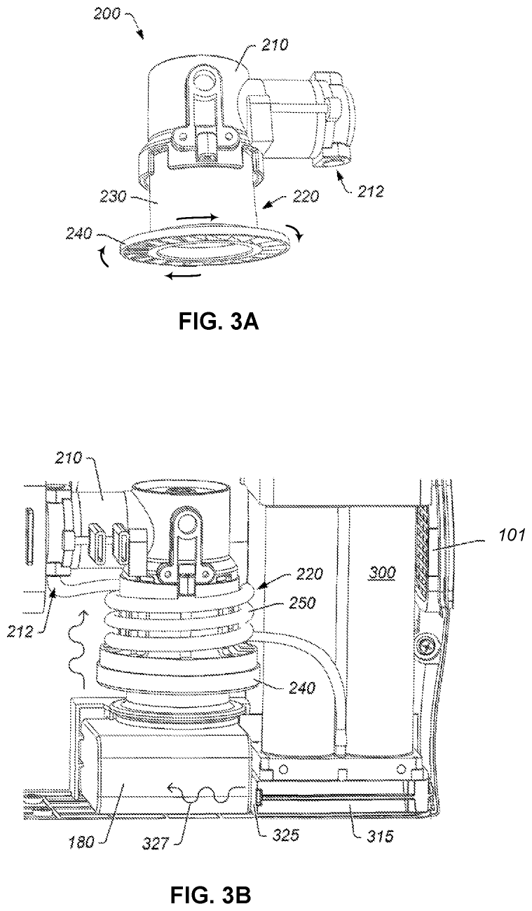

In some implementations, compression system 200 includes one or more compressors. In another implementation, compression system 200 includes a single compressor, coupled to all of the canisters of canister system 300. Turning to FIGS. 3A and 3B, a compression system 200 is depicted that includes compressor 210 and motor 220. Motor 220 is coupled to compressor 210 and provides an operating force to the compressor to operate the compression mechanism. For example, motor 220 may be a motor providing a rotating component that causes cyclical motion of a component of the compressor that compresses air. When compressor 210 is a piston type compressor, motor 220 provides an operating force which causes the piston of compressor 210 to be reciprocated. Reciprocation of the piston causes compressed air to be produced by compressor 210. The pressure of the compressed air is, in part, estimated by the speed that the compressor is operated at, (e.g., how fast the piston is reciprocated). Motor 220, therefore, may be a variable speed motor that is operable at various speeds to dynamically control the pressure of air produced by compressor 210.

In one implementation, compressor 210 includes a single head wobble type compressor having a piston. Other types of compressors may be used such as diaphragm compressors and other types of piston compressors. Motor 220 may be a DC or AC motor and provides the operating power to the compressing component of compressor 210. Motor 220, in an implementation, may be a brushless DC motor. Motor 220 may be a variable speed motor capable of operating the compressing component of compressor 210 at variable speeds. Motor 220 may be coupled to controller 400, as depicted in FIG. 1, which sends operating signals to the motor to control the operation of the motor. For example, controller 400 may send signals to motor 220 to: turn the motor on, turn motor the off, and set the operating speed of motor.

Compression system 200 inherently creates substantial heat. Heat is caused by the consumption of power by motor 220 and the conversion of power into mechanical motion. Compressor 210 generates heat due to the increased resistance to movement of the compressor components by the air being compressed. Heat is also inherently generated due to adiabatic compression of the air by compressor 210. Thus, the continual pressurization of air produces heat in the enclosure. Additionally, power supply 180 may produce heat as power is supplied to compression system 200. Furthermore, users of the oxygen concentrator may operate the device in unconditioned environments (e.g., outdoors) at potentially higher ambient temperatures than indoors, thus the incoming air will already be in a heated state.

Heat produced inside oxygen concentrator 100 can be problematic. Lithium ion batteries are generally employed as a power source for oxygen concentrators due to their long life and light weight. Lithium ion battery packs, however, are dangerous at elevated temperatures and safety controls are employed in oxygen concentrator 100 to shutdown the system if dangerously high power supply temperatures are detected. Additionally, as the internal temperature of oxygen concentrator 100 increases, the amount of oxygen generated by the concentrator may decrease. This is due, in part, to the decreasing amount of oxygen in a given volume of air at higher temperatures. If the amount of produced oxygen drops below a predetermined amount, the oxygen concentrator 100 may automatically shut down.

Because of the compact nature of oxygen concentrators, dissipation of heat can be difficult. Solutions typically involve the use of one or more fans to create a flow of cooling air through the enclosure. Such solutions, however, require additional power from the power supply and thus shorten the portable usage time of the oxygen concentrator. In an implementation, a passive cooling system may be used that takes advantage of the mechanical power produced by motor 220. Referring to FIGS. 3A and 3B, compression system 200 includes motor 220 having an external rotating armature 230. Specifically, armature 230 of motor 220 (e.g. a DC motor) is wrapped around the stationary field that is driving the armature. Since motor 220 is a large contributor of heat to the overall system it is helpful to pull heat off of the motor and sweep it out of the enclosure. With the external high speed rotation, the relative velocity of the major component of the motor and the air in which it exists is very high. The surface area of the armature is larger if externally mounted than if it is internally mounted. Since the rate of heat exchange is proportional to the surface area and the square of the velocity, using a larger surface area armature mounted externally increases the ability of heat to be dissipated from motor 220. The gain in cooling efficiency by mounting the armature externally, allows the elimination of one or more cooling fans, thus reducing the weight and power consumption while maintaining the interior of the oxygen concentrator within the appropriate temperature range. Additionally, the rotation of the externally mounted armature creates movement of air proximate to the motor to create additional cooling.

Moreover, an external rotating armature may help the efficiency of the motor, allowing less heat to be generated. A motor having an external armature operates similar to the way a flywheel works in an internal combustion engine. When the motor is driving the compressor, the resistance to rotation is low at low pressures. When the pressure of the compressed air is higher, the resistance to rotation of the motor is higher. As a result, the motor does not maintain consistent ideal rotational stability, but instead surges and slows down depending on the pressure demands of the compressor. This tendency of the motor to surge and then slow down is inefficient and therefore generates heat. Use of an external armature adds greater angular momentum to the motor which helps to compensate for the variable resistance experienced by the motor. Since the motor does not have to work as hard, the heat produced by the motor may be reduced.

In an implementation, cooling efficiency may be further increased by coupling an air transfer device 240 to external rotating armature 230. In an implementation, air transfer device 240 is coupled to the external armature 230 such that rotation of the external armature causes the air transfer device to create an airflow that passes over at least a portion of the motor. In an implementation, air transfer device includes one or more fan blades coupled to the armature. In an implementation, a plurality of fan blades may be arranged in an annular ring such that the air transfer device acts as an impeller that is rotated by movement of the external rotating armature. As depicted in FIGS. 3A and 3B, air transfer device 240 may be mounted to an outer surface of the external armature 230, in alignment with the motor. The mounting of the air transfer device to the armature allows airflow to be directed toward the main portion of the external rotating armature, providing a cooling effect during use. In an implementation, the air transfer device directs air flow such that a majority of the external rotating armature is in the air flow path.

Further, referring to FIGS. 3A and 3B, air pressurized by compressor 210 exits compressor 210 at compressor outlet 212. A compressor outlet conduit 250 is coupled to compressor outlet 212 to transfer the compressed air to canister system 300. As noted previously, compression of air causes an increase in the temperature of the air. This increase in temperature can be detrimental to the efficiency of the oxygen concentrator. In order to reduce the temperature of the pressurized air, compressor outlet conduit 250 is placed in the air flow path produced by air transfer device 240. At least a portion of compressor outlet conduit 250 may be positioned proximate to motor 220. Thus, airflow, created by air transfer device, may contact both motor 220 and compressor outlet conduit 250. In one implementation, a majority of compressor outlet conduit 250 is positioned proximate to motor 220. In an implementation, the compressor outlet conduit 250 is coiled around motor 220, as depicted in FIG. 3B.

In an implementation, the compressor outlet conduit 250 is composed of a heat exchange metal. Heat exchange metals include, but are not limited to, aluminum, carbon steel, stainless steel, titanium, copper, copper-nickel alloys or other alloys formed from combinations of these metals. Thus, compressor outlet conduit 250 can act as a heat exchanger to remove heat that is inherently caused by compression of the air. By removing heat from the compressed air, the number of molecules in a given volume at a given pressure is increased. As a result, the amount of oxygen that can be generated by each canister during each pressure swing cycle may be increased.

The heat dissipation mechanisms described herein are either passive or make use of elements required for the oxygen concentrator 100. Thus, for example, dissipation of heat may be increased without using systems that require additional power. By not requiring additional power, the run-time of the battery packs may be increased and the size and weight of the oxygen concentrator may be minimized. Likewise, use of an additional box fan or cooling unit may be eliminated. Eliminating such additional features reduces the weight and power consumption of the oxygen concentrator.

As discussed above, adiabatic compression of air causes the air temperature to increase. During venting of a canister in canister system 300, the pressure of the gas being released from the canisters decreases. The adiabatic decompression of the gas in the canister causes the temperature of the gas to drop as it is vented. In an implementation, the cooled vented gases from canister system 300 are directed toward power supply 180 and toward compression system 200. In an implementation, base 315 of compression system 300 receives the vented gases from the canisters. The vented gases 327 are directed through base 315 toward outlet 325 of the base and toward power supply 180. The vented gases, as noted, are cooled due to decompression of the gases and therefore passively provide cooling to the power supply. When the compression system is operated, the air transfer device will gather the cooled vented gases and direct the gases toward the motor of compression system 200. Fan 172 may also assist in directing the vented gas across compression system 200 and out of the housing 170. In this manner, additional cooling may be obtained without requiring any further power requirements from the battery.

### Outlet System

An outlet system, coupled to one or more of the canisters, includes one or more conduits for providing oxygen enriched gas to a user. In an implementation, oxygen enriched gas produced in either of canisters 302 and 304 is collected in accumulator 106 through check valves 142 and 144, respectively, as depicted schematically in FIG. 1. The oxygen enriched gas leaving the canisters may be collected in an oxygen accumulator 106 prior to being provided to a user. In some implementations, a tube may be coupled to the accumulator 106 to provide the oxygen enriched gas to the user. Oxygen enriched gas may be provided to the user through an airway delivery device that transfers the oxygen enriched gas to the user's mouth and/or nose. In an implementation, an outlet may include a tube that directs the oxygen toward a user's nose and/or mouth that may not be directly coupled to the user's nose.

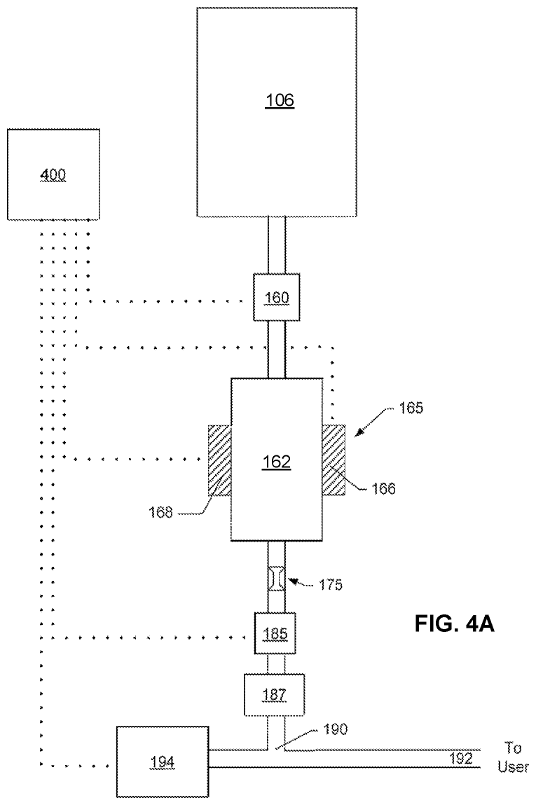

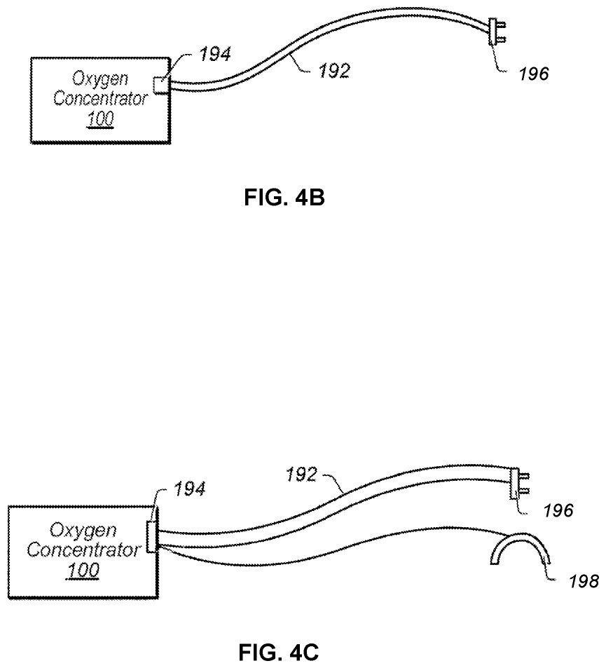

Turning to FIG. 4A, a schematic diagram of an implementation of an outlet system for an oxygen concentrator is shown. A supply valve 160 may be coupled to outlet tube to control the release of the oxygen enriched gas from accumulator 106 to the user. In an implementation, supply valve 160 is an electromagnetically actuated plunger valve. Supply valve 160 is actuated by controller 400 to control the delivery of oxygen enriched gas to a user. Actuation of supply valve 160 is not timed or synchronized to the pressure swing adsorption process. Instead, actuation is synchronized to the user's breathing as described below. In some implementations, supply valve 160 may have continuously-valued actuation to establish a clinically effective amplitude profile for providing oxygen enriched gas.

Oxygen enriched gas in accumulator 106 passes through supply valve 160 into expansion chamber 162 as depicted in FIG. 4A. In an implementation, expansion chamber may include one or more devices capable of being used to determine an oxygen concentration of gas passing through the chamber. Oxygen enriched gas in expansion chamber 162 builds briefly, through release of gas from accumulator by supply valve 160, and then is bled through a small orifice flow restrictor 175 to a flow rate sensor 185 and then to particulate filter 187. Flow restrictor 175 may be a 0.025 D flow restrictor. Other flow restrictor types and sizes may be used. In some implementations, the diameter of the air pathway in the housing may be restricted to create restricted gas flow. Flow rate sensor 185 may be any sensor capable of estimating the rate of gas flowing through the conduit. Particulate filter 187 may be used to filter bacteria, dust, granule particles, etc., prior to delivery of the oxygen enriched gas to the user. The oxygen enriched gas passes through filter 187 to connector 190 which sends the oxygen enriched gas to the user via delivery conduit 192 and to pressure sensor 194.

The fluid dynamics of the outlet pathway, coupled with the programmed actuations of supply valve 160, may result in a bolus of oxygen being provided at the correct time and with an amplitude profile that assures rapid delivery into the user's lungs without excessive waste. If the bolus can be delivered in this manner, there may be a linear relationship between the prescribed continuous flow rate and the therapeutically equivalent bolus volume required in pulsed delivery mode for a user at rest with a given breath pattern. For example, the total volume of the bolus required to emulate continuous-flow prescriptions may be equal to 11 mL for each LPM of prescribed continuous flow rate, i.e., 11 mL for a prescription of 1 LPM; 22 mL for a prescription of 2 LPM; 33 mL for a prescription of 3 LPM; 44 mL for a prescription of 4 LPM; 55 mL for a prescription of 5 LPM; etc. This amount is generally referred to as the LPM equivalent bolus volume. It should be understood that the LPM equivalent may vary between oxygen concentrators due to differences in construction design, tubing size, chamber size, etc. The LPM equivalent will also vary depending on the user's breath pattern (e.g. breathing rate).

Expansion chamber 162 may include one or more oxygen sensors adapted to determine an oxygen concentration of gas passing through the chamber. In an implementation, the oxygen concentration of gas passing through expansion chamber 162 is estimated using an oxygen sensor 165. An oxygen sensor is a device capable of detecting oxygen in a gas. Examples of oxygen sensors include, but are not limited to, ultrasonic oxygen sensors, electrical oxygen sensors, and optical oxygen sensors. In one implementation, oxygen sensor 165 is an ultrasonic oxygen sensor that includes an ultrasonic emitter 166 and an ultrasonic receiver 168. In some implementations, ultrasonic emitter 166 may include multiple ultrasonic emitters and ultrasonic receiver 168 may include multiple ultrasonic receivers. In implementations having multiple emitters/receivers, the multiple ultrasonic emitters and multiple ultrasonic receivers may be axially aligned (e.g., across the gas mixture flow path which may be perpendicular to the axial alignment).

In use, an ultrasonic sound wave (from emitter 166) may be directed through oxygen enriched gas disposed in chamber 162 to receiver 168. Ultrasonic sensor assembly may be configured to detect the speed of sound through the gas mixture to determine the composition of the gas mixture (e.g., the speed of sound is different in nitrogen and oxygen). In a mixture of the two gases, the speed of sound through the mixture may be an intermediate value proportional to the relative amounts of each gas in the mixture. In use, the sound at the receiver 168 is slightly out of phase with the sound sent from emitter 166. This phase shift is due to the relatively slow velocity of sound through a gas medium as compared with the relatively fast speed of the electronic pulse through wire. The phase shift, then, is proportional to the distance between the emitter and the receiver and the speed of sound through the expansion chamber. The density of the gas in the chamber affects the speed of sound through the chamber and the density is proportional to the ratio of oxygen to nitrogen in the chamber. Therefore, the phase shift can be used to measure the concentration of oxygen in the expansion chamber. In this manner the relative concentration of oxygen in the accumulator may be estimated as a function of one or more properties of a detected sound wave traveling through the accumulator.

In some implementations, multiple emitters 166 and receivers 168 may be used. The readings from the emitters 166 and receivers 168 may be averaged to cancel errors that may be inherent in turbulent flow systems. In some implementations, the presence of other gases may also be detected by measuring the transit time and comparing the measured transit time to predetermined transit times for other gases and/or mixtures of gases.

The sensitivity of the ultrasonic sensor system may be increased by increasing the distance between the emitter 166 and receiver 168, for example to allow several sound wave cycles to occur between emitter 166 and the receiver 168. In some implementations, if at least two sound cycles are present, the influence of structural changes of the transducer may be reduced by measuring the phase shift relative to a fixed reference at two points in time. If the earlier phase shift is subtracted from the later phase shift, the shift caused by thermal expansion of expansion chamber 162 may be reduced or cancelled. The shift caused by a change of the distance between the emitter 166 and receiver 168 may be approximately the same at the measuring intervals, whereas a change owing to a change in oxygen concentration may be cumulative. In some implementations, the shift measured at a later time may be multiplied by the number of intervening cycles and compared to the shift between two adjacent cycles. Further details regarding sensing of oxygen in the expansion chamber may be found, for example, in U.S. Published Patent Application No. 2009-0065007, published Mar. 12, 2009, and entitled “Oxygen Concentrator Apparatus and Method, which is incorporated herein by reference.

Flow rate sensor 185 may be used to determine the flow rate of gas flowing through the outlet system. Flow rate sensors that may be used include, but are not limited to: diaphragm/bellows flow meters; rotary flow meters (e.g. Hall effect flow meters); turbine flow meters; orifice flow meters; and ultrasonic flow meters. Flow rate sensor 185 may be coupled to controller 400. The rate of gas flowing through the outlet system may be an indication of the breathing volume of the user. Changes in the flow rate of gas flowing through the outlet system may also be used to determine a breathing rate of the user. Controller 400 may generate a control signal or trigger signal to control actuation of supply valve 160. Such control of actuation of the supply valve may be based on the breathing rate and/or breathing volume of the user, as estimated by flow rate sensor 185 and/or may be based on other sensor signals, such as by implementing any of the control methodologies described herein concerning bolus release.

In some implementations, ultrasonic sensor system 165 and, for example, flow rate sensor 185 may provide a measurement of an actual amount of oxygen being provided. For example, flow rate sensor 185 may measure a volume of gas (based on flow rate) provided and ultrasonic sensor system 165 may provide the concentration of oxygen of the gas provided. These two measurements together may be used by controller 400 to determine an approximation of the actual amount of oxygen provided to the user.

Oxygen enriched gas passes through flow rate sensor 185 to filter 187. Filter 187 removes bacteria, dust, granule particles, etc., prior to providing the oxygen enriched gas to the user. The filtered oxygen enriched gas passes through filter 187 to connector 190. Connector 190 may be a “Y” connector coupling the outlet of filter 187 to pressure sensor 194 and delivery conduit 192. Pressure sensor 194 may be used to monitor the pressure of the gas passing through conduit 192 to the user. In some implementations, pressure sensor 194 is configured to generate a signal that is proportional to the amount of positive or negative pressure applied to a sensing surface. Changes in pressure, sensed by pressure sensor 194, may be used to determine a breathing rate of a user, as well as the onset of inhalation (also referred to as the trigger instant) as described below. Controller 400 may control actuation of supply valve 160 based on the breathing rate and/or onset of inhalation of the user. In an implementation, controller 400 may control actuation of supply valve 160 based on information provided by either or both of the flow rate sensor 185 and the pressure sensor 194.

Oxygen enriched gas may be provided to a user through conduit 192. In an implementation, conduit 192 may be a silicone tube. Conduit 192 may be coupled to a user using an airway delivery device 196, as depicted in FIGS. 4B and 4C. Airway delivery device 196 may be any device capable of providing the oxygen enriched gas to nasal cavities or oral cavities. Examples of airway coupling members include, but are not limited to: nasal masks, nasal pillows, nasal prongs, nasal cannulas, and mouthpieces. A nasal cannula airway delivery device is depicted in FIG. 4B. During use, oxygen enriched gas from oxygen concentrator 100 is provided to the user through conduit 192 and airway delivery device 196. Airway delivery device 196 is positioned proximate to a user's airway (e.g., proximate to the user's mouth and or nose) to allow delivery of the oxygen enriched gas to the user while allowing the user to breathe air from the surroundings.

In an alternate implementation, a mouthpiece may be used to provide oxygen enriched gas to the user. As shown in FIG. 4C, a mouthpiece 198 may be coupled to oxygen concentrator 100. Mouthpiece 198 may be the only device used to provide oxygen enriched gas to the user, or a mouthpiece may be used in combination with a nasal delivery device (e.g., a nasal cannula). As depicted in FIG. 4C, oxygen enriched gas may be provided to a user through both a nasal airway delivery device 196 and a mouthpiece 198.

Mouthpiece 198 is removably positionable in a user's mouth. In one implementation, mouthpiece 198 is removably couplable to one or more teeth in a user's mouth. During use, oxygen enriched gas is directed into the user's mouth via the mouthpiece. Mouthpiece 198 may be a night guard mouthpiece which is molded to conform to the user's teeth. Alternatively, mouthpiece may be a mandibular repositioning device. In an implementation, at least a majority of the mouthpiece is positioned in a user's mouth during use.

During use, oxygen enriched gas may be directed to mouthpiece 198 when a change in pressure is detected proximate to the mouthpiece. In one implementation, mouthpiece 198 may be coupled to a pressure sensor. When a user inhales air through the user's mouth, pressure sensor 194 may detect a drop in pressure proximate to the mouthpiece. Controller 400 of oxygen concentrator 100 may control release of a bolus of oxygen enriched gas to the user at the onset of inhalation such as by generating a bolus release control signal or trigger signal that controls a supply valve 160.

During typical breathing of an individual, inhalation may occur through the nose, through the mouth or through both the nose and the mouth. Furthermore, breathing may change from one passageway to another depending on a variety of factors. For example, during more active activities, a user may switch from breathing through their nose to breathing through their mouth, or breathing through their mouth and nose. A system that relies on a single mode of delivery (either nasal or oral), may not function properly if breathing through the monitored pathway is stopped. For example, if a nasal cannula is used to provide oxygen enriched gas to the user, an inhalation sensor (e.g., a pressure sensor or flow rate sensor) is coupled to the nasal cannula to determine the onset of inhalation. If the user stops breathing through their nose, and switches to breathing through their mouth, the oxygen concentrator 100 may not know when to provide the oxygen enriched gas since there is no feedback from the nasal cannula. Under such circumstances, oxygen concentrator 100 may increase the flow rate and/or increase the frequency of providing oxygen enriched gas until the inhalation sensor detects an inhalation by the user. If the user switches between breathing modes often, the default mode of providing oxygen enriched gas may cause the oxygen concentrator 100 to work harder, limiting the portable usage time of the system.

In an implementation, a mouthpiece 198 is used in combination with an airway delivery device 196 (e.g., a nasal cannula) to provide oxygen enriched gas to a user, as depicted in FIG. 4C. Both mouthpiece 198 and airway delivery device 196 are coupled to an inhalation sensor. In one implementation, mouthpiece 198 and airway delivery device 196 are coupled to the same inhalation sensor. In an alternate implementation, mouthpiece 198 and airway delivery device 196 are coupled to different inhalation sensors. In either implementation, inhalation sensor(s) may now detect the onset of inhalation from either the mouth or the nose. Oxygen concentrator 100 may be configured to provide oxygen enriched gas to the device (i.e. mouthpiece 198 or airway delivery device 196) proximate to which the onset of inhalation was detected. Alternatively, oxygen enriched gas may be provided to both mouthpiece 198 and the airway delivery device 196 if onset of inhalation is detected proximate either device. The use of a dual delivery system, such as depicted in FIG. 4C may be particularly useful for users when they are sleeping and may switch between nose breathing and mouth breathing without conscious effort.

### Controller System

Operation of oxygen concentrator 100 may be performed automatically using an internal controller 400 coupled to various components of the oxygen concentrator 100, as described herein. Controller 400 includes one or more processors 410 and internal memory 420, as depicted in FIG. 1. Methods used to operate and monitor oxygen concentrator 100 may be implemented by program instructions stored in memory 420 or a carrier medium coupled to controller 400, and executed by one or more processors 410. A memory medium may include any of various types of memory devices or storage devices. The term “memory medium” is intended to include an installation medium, e.g., a Compact Disc Read Only Memory (CD-ROM), floppy disks, or tape device; a computer system memory or random access memory such as Dynamic Random Access Memory (DRAM), Double Data Rate Random Access Memory (DDR RAM), Static Random Access Memory (SRAM), Extended Data Out Random Access Memory (EDO RAM), Random Access Memory (RAM), etc.; or a non-volatile memory such as a magnetic media, e.g., a hard drive, or optical storage. The memory medium may comprise other types of memory as well, or combinations thereof. In addition, the memory medium may be located in a first computer in which the programs are executed, or may be located in a second different computer that connects to the first computer over a network, such as the Internet. In the latter instance, the second computer may provide program instructions to the first computer for execution. The term “memory medium” may include two or more memory mediums that may reside in different locations, e.g., in different computers that are connected over a network.

In some implementations, controller 400 includes processor 410 that includes, for example, one or more field programmable gate arrays (FPGAs), microcontrollers, etc. included on a circuit board disposed in oxygen concentrator 100. Processor 410 is capable of executing programming instructions stored in memory 420. In some implementations, programming instructions may be built into processor 410 such that a memory external to the processor may not be separately accessed (i.e., the memory 420 may be internal to the processor 410).

Processor 410 may be coupled to various components of oxygen concentrator 100, including, but not limited to compression system 200, one or more of the valves used to control fluid flow through the system (e.g., valves 122, 124, 132, 134, 152, 154, 160), oxygen sensor 165, pressure sensor 194, flow rate sensor 185, temperature sensors (not shown), fans, and any other component that may be electrically controlled. In some implementations, a separate processor (and/or memory) may be coupled to one or more of the components.

Controller 400 is configured (e.g., programmed) to operate oxygen concentrator 100 and is further configured to monitor the oxygen concentrator 100 for malfunction states. For example, in one implementation, controller 400 is programmed to trigger an alarm if the system is operating and no breathing is detected by the user for a predetermined amount of time. For example, if controller 400 does not detect a breath for a period of 75 seconds, an alarm LED may be lit and/or an audible alarm may be sounded. If the user has truly stopped breathing, for example, during a sleep apnea episode, the alarm may be sufficient to awaken the user, causing the user to resume breathing. The action of breathing may be sufficient for controller 400 to reset this alarm function. Alternatively, if the system is accidentally left on when delivery conduit 192 is removed from the user, the alarm may serve as a reminder for the user to turn oxygen concentrator 100 off

Controller 400 is further coupled to oxygen sensor 165, and may be programmed for continuous or periodic monitoring of the oxygen concentration of the oxygen enriched gas passing through expansion chamber 162. A minimum oxygen concentration threshold may be programmed into controller 400, such that the controller lights an LED visual alarm and/or an audible alarm to warn the user of the low concentration of oxygen.

Controller 400 is also coupled to internal power supply 180 and is capable of monitoring the level of charge of the internal power supply. A minimum voltage and/or current threshold may be programmed into controller 400, such that the controller lights an LED visual alarm and/or an audible alarm to warn the user of low power condition. The alarms may be activated intermittently and at an increasing frequency as the battery approaches zero usable charge.

Further functions that may be implemented with or by the controller 400 are described in detail in other sections of this disclosure.

### Outer Housing—Control Panel

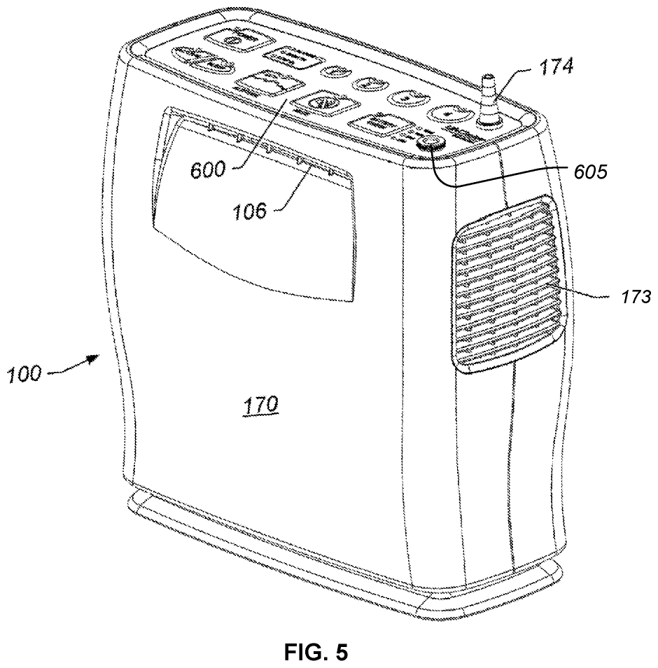

FIG. 5 depicts an implementation of an outer housing 170 of an oxygen concentrator 100. In some implementations, outer housing 170 may be comprised of a light-weight plastic. Outer housing includes compression system inlets 105, cooling system passive inlet 101 and outlet 173 at each end of outer housing 170, outlet port 174, and control panel 600. Inlet 101 and outlet 173 allow cooling air to enter the housing, flow through the housing, and exit the interior of housing 170 to aid in cooling of the oxygen concentrator 100. Compression system inlets 105 allow air to enter the compression system. Outlet port 174 is used to attach a conduit to provide oxygen enriched gas produced by the oxygen concentrator 100 to a user.

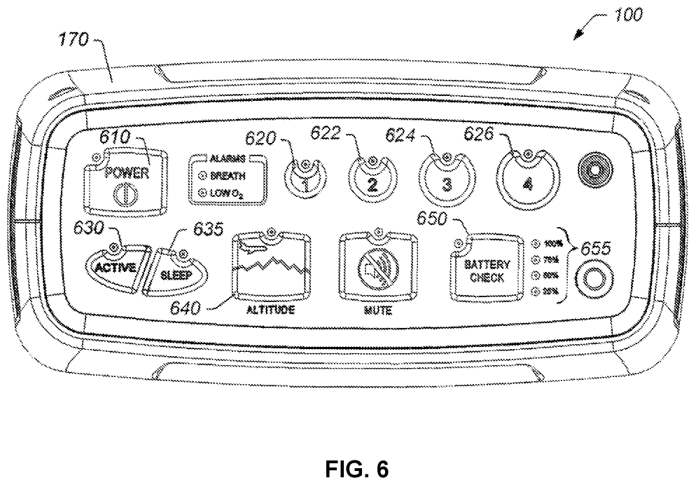

Control panel 600 serves as an interface between a user and controller 400 to allow the user to initiate predetermined operation modes of the oxygen concentrator 100 and to monitor the status of the system. Charging input port 605 may be disposed in control panel 600. FIG. 6 depicts an implementation of control panel 600.

In some implementations, control panel 600 may include buttons to activate various operation modes for the oxygen concentrator 100. For example, control panel may include power button 610, dosage buttons 620 to 626, active mode button 630, sleep mode button 635, and a battery check button 650. In some implementations, one or more of the buttons may have a respective LED that may illuminate when the respective button is pressed (and may power off when the respective button is pressed again). Power button 610 may power the system on or off If the power button is activated to turn the system off, controller 400 may initiate a shutdown sequence to place the system in a shutdown state (e.g., a state in which both canisters are pressurized). Dosage buttons 620, 622, 624, and 626 allow the prescribed continuous flow rate of oxygen enriched gas to be selected (e.g., 1 LPM by button 620, 2 LPM by button 622, 3 LPM by button 624, and 4 LPM by button 626). Altitude button 640 may be selected when a user is going to be in a location at a higher elevation than the oxygen concentrator 100 is regularly used by the user. The adjustments made by the oxygen concentrator 100 in response to activating altitude mode are described in more detail herein.

Battery check button 650 initiates a battery check routine in the oxygen concentrator 100 which results in a relative battery power remaining LED 655 being illuminated on control panel 600.

A user may have a low breathing rate or depth if relatively inactive (e.g., asleep, sitting, etc.) as estimated by comparing the detected breathing rate or depth to a threshold. The user may have a high breathing rate or depth if relatively active (e.g., walking, exercising, etc.). An active/sleep mode may be estimated automatically and/or the user may manually indicate a respective active or sleep mode by pressing button 630 for active mode and button 635 for sleep mode. The adjustments made by the oxygen concentrator 100 in response to activating active mode or sleep mode are described in more detail herein.

### Methods of Controlling Release of Oxygen Enriched Gas

The main use of an oxygen concentrator 100 is to provide supplemental oxygen to a user. Generally, the flow rate of supplemental oxygen to be provided is estimated by a physician. Typical prescribed continuous flow rates of supplemental oxygen may range from about 1 LPM to up to about 10 LPM. The most commonly prescribed continuous flow rates are 1 LPM, 2 LPM, 3 LPM, and 4 LPM. Generally, in a pulsed oxygen device, oxygen enriched gas is provided to the user in synchrony with the breathing cycle at sufficient volume to emulate the continuous flow rate prescribed for the user. As used herein the term “breathing cycle” refers to an inhalation followed by an exhalation.

In order to minimize the amount of oxygen enriched gas that is needed to be produced to emulate the prescribed continuous flow rate, controller 400 may be programmed to time release of the oxygen enriched gas with the user's inhalations, according to a therapy mode known as pulsed oxygen delivery (POD) or demand oxygen delivery. Releasing a bolus of oxygen enriched gas to the user as the user inhales may prevent unnecessary oxygen generation (further reducing power requirements) by not releasing oxygen, for example, when the user is exhaling. Reducing the amount of oxygen required may effectively reduce the amount of air compression needed by oxygen concentrator 100 (and subsequently may reduce the power demand from the compressors).

Oxygen enriched gas produced by oxygen concentrator 100 is stored in an oxygen accumulator 106 and released to the user as the user inhales. The amount of oxygen enriched gas provided by the oxygen concentrator 100 is controlled, in part, by supply valve 160. In an implementation, supply valve 160 is opened for a sufficient amount of time to provide the appropriate amount of oxygen enriched gas, as estimated by controller 400, to the user. In order to minimize the amount of oxygen required to emulate the prescribed continuous flow rate of a user, the oxygen enriched gas may be provided as a bolus when the onset of a user's inhalation is detected. For example, the bolus of oxygen enriched gas may be provided in the first few milliseconds of a user's inhalation.

In an implementation, pressure sensor 194 may be used to determine the onset of inhalation by the user. For example, the user's inhalation may be detected by using pressure sensor 194. In use, conduit 192 for providing oxygen enriched gas is coupled to a user's nose and/or mouth through the airway delivery device 196 and/or 198. The pressure in conduit 192 is therefore representative of the user's airway pressure. At the onset of an inhalation, the user begins to draw air into their body through the nose and/or mouth. As the air is drawn in, a negative pressure is generated at the end of the conduit, due, in part, to the venturi action of the air being drawn across the end of the conduit. Controller 400 analyses the pressure signal from the pressure sensor 194 to detect a drop in pressure, to indicate the onset of inhalation. Upon detection of the onset of inhalation, supply valve 160 is opened, such as in response to a generated control signal for the valve, to release a bolus of oxygen enriched gas from the accumulator 106. A positive change or rise in the pressure indicates an exhalation by the user and is generally a time that release of oxygen enriched gas is discontinued. In one implementation, when a positive pressure change is sensed, supply valve 160 is closed, such as in response to the generated control signal for the valve, until the next onset of inhalation. Alternatively, supply valve 160 may be closed after a predetermined interval known as the bolus duration. By measuring the intervals between adjacent onsets of inhalation, the user's breathing rate may be estimated. By measuring the intervals between onsets of inhalation and the following onsets of exhalation, the user's inspiratory time may be estimated.

In other implementations, the pressure sensor 194 may be located in a sensing conduit that is in pneumatic communication with the user's airway, but separate from the delivery conduit 192. In such implementations the pressure signal from the pressure sensor 194 is therefore also representative of the user's airway pressure.

In some implementations, the sensitivity of the pressure sensor 194 may be affected by the physical distance of the pressure sensor 194 from the user, especially if the pressure sensor 194 is located in oxygen concentrator 100 and the pressure difference is detected through the conduit 192 coupling the oxygen concentrator 100 to the user. In some implementations, the pressure sensor 194 may be placed in the airway delivery device 196 used to provide the oxygen enriched gas to the user. A signal from the pressure sensor 194 may be provided to controller 400 in the oxygen concentrator 100 electronically via a wire or through telemetry such as through Bluetooth™ or other wireless technology.

In some implementations, if the user's current activity level, such as estimated using the detected user's breathing rate, exceeds a predetermined threshold, controller 400 may implement an alarm (e.g., visual and/or audio) to warn the user that the current breathing rate is exceeding the delivery capacity of the oxygen concentrator 100. For example, the threshold may be set at 40 breaths per minute (BPM).

### Triggering POD

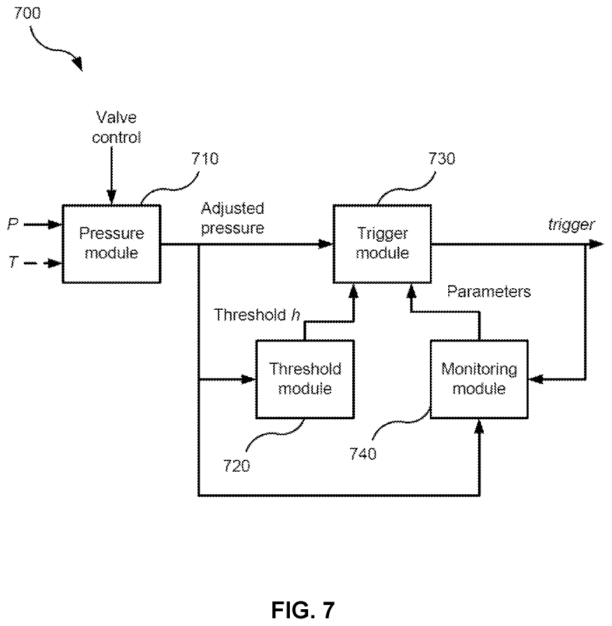

FIG. 7 is a block diagram illustrating an example adaptive triggering system 700 configured to trigger the release of a bolus of oxygen from the oxygen concentrator 100 for delivery to a user according to one implementation of the present technology. The various modules 710, 720, 730, and 740 of the system 700 may be implemented as processing components of the system or otherwise encoded as program instructions stored in memory 420 and executed by the controller 400.

While the functionality of the various modules may be as set out below, in other implementations the functionality may be partitioned differently between the modules.

The system 700 may include a pressure module 710. The pressure module 710 may be configured to receive as input any or all of: a raw pressure signal P from the pressure sensor 194, the valve control signal pulse generated by the controller 400 to control the supply valve 160, and (optionally) a temperature signal T from a temperature sensor in the oxygen concentrator 100. The function of the pressure module 710 is to adjust the raw pressure signal so that it more accurately represents the user's airway pressure. The pressure module 710 may do this by removing the pressure pulse(s) or pressure effect(s) that is/are contained in the raw pressure signal as a consequence of each release of a bolus, in implementations where the pressure sensor 194 is in the delivery conduit 192. The incorporation of the temperature signal Tallows the pressure module 710 to compensate for variations in temperature by removing any offset drift (thermal or other) in the raw pressure signal P that may be caused by those variations if the pressure sensor 194 is temperature sensitive. The pressure module 710 also performs noise reduction filtering on the raw pressure signal P. The output of the pressure module 710 is an “adjusted” pressure signal P_{adj} as a function of time t. The pressure module 710 will be described in more detail below.

The system 700 may further include a threshold module 720. The function of the threshold module 720 is to monitor the adjusted pressure signal P_{adj} from the pressure module 710 and repeatedly determine an appropriate trigger threshold h as a function of time t. The threshold module 720 will be described in more detail below.

The system 700 may further include a trigger module 730. The function of the trigger module 730 is to apply the trigger threshold h from the threshold module 720 to the adjusted pressure signal P_{adj} from the pressure module 710 to generate a (e.g., Boolean) signal trigger, that may be taken as an indication of an onset of inhalation. The signal trigger may be used as a bolus release control or trigger signal mentioned above. The trigger module 730 may also optionally achieve its function using one or more breathing parameters received from monitoring module 740. The trigger module 730 will be described in more detail below.

The system 700 may further include a monitoring module 740. One function of the monitoring module 740 is to monitor the user's airway pressure (such as by monitoring of the adjusted pressure signal P_{adj} from the pressure module 710 that is representative of user's airway pressure) and the trigger signal from the trigger module 730. The monitoring module 740 may calculate one or more breathing parameters of the user, e.g. the user's breathing rate R, such as based on pressure and/or inhalation onset information (e.g., based on the adjusted pressure signal and/or the trigger signal). One or more of these breathing parameters may be passed to the trigger module 730. The breathing parameters generated by the monitoring module 740 may also be provided to modules external to the triggering system 700 including bolus adjustment schemes and user data reporting. The monitoring module 740 will be described in more detail below.

In some implementations, the modules 710 to 740 operate in synchrony at a predetermined “real time” sample rate ranging from 100 Hz to 1 kHz, except where described below. The pressure signal P and temperature signal T are generated by their respective sensors at, at least, the real time rate.

In the following description, a sign convention is described such that the pressure signal P is taken to be negative during inhalation and positive during exhalation. However, such signaling can, in some implementations, be inverted and/or otherwise adjusted to maintain a particular range (e.g., positive values or negative values) and applied to achieve the same result.

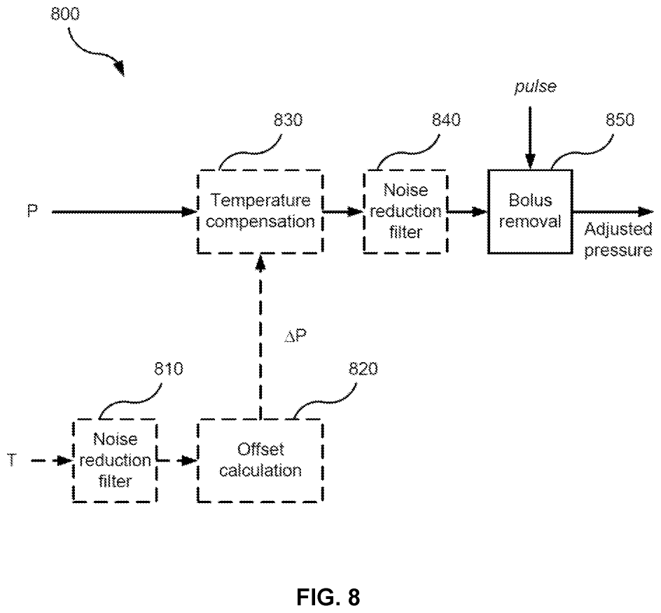

FIG. 8 is a block diagram illustrating one implementation of a pressure module 800 comparable to the pressure module 710 of the system 700 of FIG. 7 according to the present technology. The pressure module 800 takes as input the raw pressure signal P from the pressure sensor 194, the valve control signal pulse generated by the controller 400 to control the supply valve 160, and (optionally) a temperature signal T from a temperature sensor in the oxygen concentrator 100. Optional elements in the pressure module 800 are shown in dashed outline.

If the temperature signal T is present, the pressure module 800 may filter the signal such as by applying a filter 810 to the temperature signal T for noise reduction. In one implementation, the filter 810 is a three-point median filter. Next, offset calculation module 820 may calculate a pressure offset ΔP as a function of the change in temperature in relation to a reference temperature T_{ref}. Such a reference temperature T_{ref} may be a temperature that is determined/measure at a previous calibration or operation (or at power-up if none has been performed) using the temperature signal or temperature sensor. In one implementation, the pressure offset ΔP is calculated as a linear function of the change in temperature:

ΔP=K(T−T_{ref}) (1)

where K is a compensation coefficient, such as for converting from units of temperature to units of pressure. In an alternative implementation, offset calculation module 820 retrieves the pressure offset ΔP from a pre-calibrated lookup table based on T and T_{ref}.

In an alternative implementation, suitable when no temperature signal T is present, the pressure offset ΔP may be estimated by monitoring the raw pressure signal P. The estimated pressure offset ΔP is that value which, when subtracted from the raw pressure signal P, would result in the raw pressure signal P being negative (indicating inhalation) for a period of average duration T_{I}, and positive (indicating inhalation) for a period of average duration T_{E}, where T_{I} and T_{E} are in a known ratio I:E of inspiratory time to expiratory time. In other words, the estimated pressure offset ΔP is chosen such that the periods of raw pressure above ΔP and the periods of raw pressure below ΔP meet a predetermined I:E ratio. The I:E ratio for most users is in the range 1.1 to 1.4. Users under respiratory duress, including COPD patients in a poor state, will have an I:E ratio closer to 1:1, while relaxed healthy users will have an I:E ratio as high as 1:4.

The raw pressure signal P may then be compensated for temperature at module 830 by applying the pressure offset ΔP to adjust the raw pressure signal, such as by subtracting the pressure offset ΔP. The pressure module 800 may then (optionally) filter the compensated pressure signal such as by applying a filter 840 to the compensated pressure to remove very short duration, large magnitude noise impulses due to switching within the electrical system of the oxygen concentrator 100. In one implementation, the filter 840 comprises a three-point median filter for this purpose. The filter 840 may also smooth the pressure signal P (e.g., the compensated pressure signal) to remove periodic device noise such as high frequency compressor and PSA noise originating from the electrical system of the oxygen concentrator 100. In one implementation, the filter 840 comprises a 24-point moving average filter for this purpose.

During bolus delivery, the user's airway pressure as sensed in the delivery conduit 192 is masked by the pressure of the bolus. The period of delivery of the bolus is approximately coincident with the release operation of the generated valve control signal. The bolus removal module 850 may therefore compute adjusted pressure values P_{adj} that are more accurately representative of the user's airway pressure during (a) the period of bolus delivery (e.g., as indicated by assertion of the valve control signal pulse) and optionally (b) a settling period thereafter. For example, a pressure pulse can temporarily elevate the pressure represented in the signal from the pressure sensor. Thus, the pressure signal may be adjusted to remove the impact or effect (e.g., pressure effect) of the bolus on the pressure signal. In some versions, such a bolus removal computation of the module 850 may involve an interpolation of pressure values between the last pressure value P₀ prior to the bolus delivery and the first pressure value P₁ after the bolus delivery and optionally the settling period. Such a settling period may optionally be set in one implementation to about 200 ms or otherwise as some time in a range of 100 to 600 ms. In one implementation, the interpolation between P₀ and P₁ is linear, according to the following equation:

$${P}_{adj} ( t ) = {P}_{0} + \frac{( t - {T}_{0} )}{T} ( {P}_{1} - {P}_{0} ) \qquad ( 2 )$$

where T₀ is the start time of bolus delivery and T is the total duration of the bolus and the settling period. At other times, P_{adj} is the (optionally) filtered (optionally) temperature-compensated pressure signal P. The settling period, which is the period between the end of a bolus and a time when the pressure signal restabilises to the patient airway pressure, may be dependent on factors including the bolus' maximum pressure and volume, the physical properties of the airway delivery device 196, and the speed of operation of the supply valve 160. In one implementation, the settling period is approximately 500 milliseconds (e.g., 501 milliseconds). In other implementations, the settling period may be estimated dynamically by monitoring the pressure signal P. In another implementation the settling period can be set by a “learn circuit” function that operates to measure such a period by determining time from delivery of a bolus to time that the pressure signal restabilizes.

In other implementations, the interpolation may be achieved by non-linear curve fitting, time-series forecasting, or respiratory signal modelling.

The adjusted pressure computation operations of system 700 may be performed in spurts. In this regard, the computation of the adjusted pressure P_{adj} may pause/wait during bolus delivery and the settling period, since P₁ might not yet be known during this delivery and/or settling period. Thus, the operation of the system 700 may be suspended during this interval. After the bolus delivery and the settling period are complete, the estimated values of the adjusted pressure signal P_{adj} may be processed at a rate faster than real time so the system 700 can catch up with real time after a short interval. If the values of the adjusted pressure signal P_{adj} are computed at a rate that is N times real time, the system 700 catches up with real time after approximately 1/(N−1)^{th} of the duration of the bolus delivery and settling period. In one implementation, N is 20.

In another implementation, no adjustment of the pressure signal during bolus delivery is performed and the period of bolus delivery is simply omitted from further processing. In such implementations, any modules are compensated for the missing period, and an interval is allowed for any filters to stabilise before resuming processing the adjusted pressure signal P_{adj}.

In some implementations, the bolus removal module 850 may be omitted. For example, the bolus removal module 850 may be omitted in implementations in which the pressure sensor 194 is located in a separate sensing conduit.

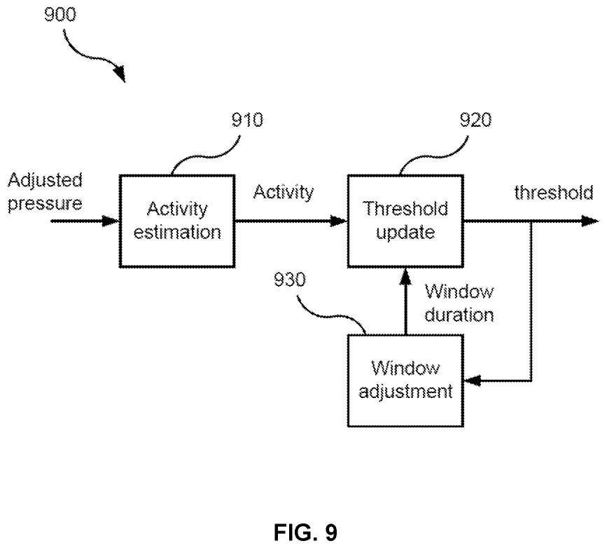

FIG. 9 is a block diagram illustrating one implementation of a theshold module 900 comparable to the threshold module 720 of the system 700 of FIG. 7 according to the present technology. As mentioned above, the function of the threshold module 900 is to monitor a pressure signal, such as the adjusted pressure signal P_{adj}, from the pressure module 710 and determine the appropriate trigger threshold h at the current time t.

The threshold module 900 may have an activity estimation module 910, or sub-module, such as one or more filter(s), configured to generate or extract an activity signal a(t) such as from an input signal such as the adjusted pressure signal P_{adj}. In contrast to a breathing parameter such as breathing rate, such a module may generate an output indicative of user activity, that is, activity other than respiration activity. For example, in one implementation, the activity estimation module 910 may be a module that applies a second-order Butterworth high-pass filter with a suitable cutoff frequency (e.g., 10 Hz). The filter and cutoff may be chosen to generate an output indicative of user activity other than respiration by omitting respiration activity from the signal. As generated in this example, higher values of a(t) typically indicate higher activity of the user, but may indicate other sources of noise as well.

The threshold update module 920 of the threshold module 900 may then adjust (e.g., repeatedly) the trigger threshold h according to the recent activity a(t). For example, the trigger threshold h may be adjusted to increase the noise immunity, i.e. decreasing its sensitivity to trigger a bolus when the activity increases (e.g., the threshold may be adjusted to be more negative). Similarly, the trigger threshold h may be adjusted to decrease the noise immunity, i.e. increasing its sensitivity to trigger a bolus when the activity decreases (e.g., the threshold may be adjusted to be less negative). Thus, the noise immunity of the adaptive trigger system 700 roughly follows the level of activity reflected in the input signal (e.g., the airway pressure signal) such as from a particular period of time of the signal (e.g., a window). For example, the threshold h may be repeatedly set as a function (e.g., a maximum, a minimum and/or average etc.) of one or more values from one or more periods of time of the input signal. In one implementation of the threshold update module 920, the trigger threshold h may be set to the maximum value amax of the activity a(t) over a window W of recent values of a(t), and may be multiplied by a scaling constant K, which may also be reversed in sign to make the trigger threshold h negative such as according to the following function:

$$\begin{matrix}h = ( - 1 ) \times K \times {a}_{max} where & ( 3 ) \\ {a}_{max} = \begin{matrix}max \\ t ∈ W\end{matrix} ( a ( t ) ) & ( 4 )\end{matrix}$$

In some implementations, the scaling constant K is fixed at 1.2.

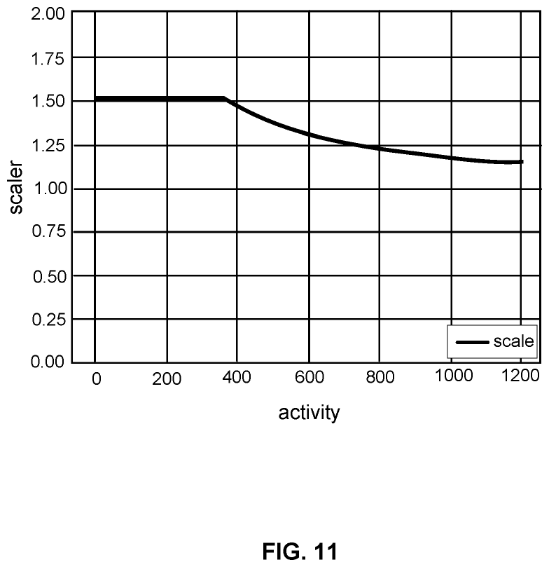

In other implementations, the scaling constant K varies dependent on (e.g., as a function of) the maximum activity aₘₐₓ. In one such implementation, for low activity levels, the scaling constant K is set to 1.5, and thereafter decreases, such as toward 1, to dampen the amplification at high levels of activity. FIG. 11 is a graph illustrating the value of the scaling constant K as a function of the maximum activity aₘₐₓ according to this implementation.

In yet other implementations, the scaling constant K varies with the user's breathing rate R, for example K=2 at low breathing rates (e.g., approximately 4 to 10 breaths per minute) reducing to 1 at high breathing rates (e.g., rates substantially higher than the low breathing rates). The intention of such implementations is to forgo immunity to noise when the resulting trigger delay (see below) would result in the bolus being released too late to be effective.

In some implementations, the threshold adjustments may be limited so that the threshold h may be maintained or limited by (e.g., to be more negative than) a minimum threshold value hmin to handle short-term pressure sensor offset error. The minimum threshold hmin is limited by sensor performance and resolution. In some implementations, the minimum threshold hmin is greater than the maximum expected short-term and uncorrected long-term offset error. Long-term offset error may be corrected by intermittent sensor calibration, e.g. upon power-up, in which case only the maximum uncorrected long-term offset error need be considered when selecting a value for the minimum threshold hmin. In one example, the minimum threshold hmin is in the range of −0.01 to −0.5 mmH₂O, e.g. −0.15 mmH₂O.

In some implementations the value of hmin is increased (in a negative sense i.e., made more negative) as a function of amount of time since the last successful calibration. This enables maximum performance when calibrated and assists optimum (but necessarily reduced) performance for devices at risk of long-term offset drift.

In some implementations, threshold adjustments may be limited so that the threshold h may be maintained or limited by (e.g., to be less negative than) a maximum threshold value hmax to reduce the chance of missed inspirations (false negatives) during periods of high noise on the adjusted pressure signal. In one such implementation, the maximum threshold value hmax may be computed as a fraction of the minimum adjusted pressure Padj.

The duration w of the window W may be fixed, e.g. at 10 seconds or other time value in a range of, for example, about 5 to 15 seconds. In this regard, the duration of the window concerns an amount of data (or values) of the activity signal that are considered in any given threshold determination. In some versions, the window may be adjustable by the window adjustment module 930 such as by adjusting the window as a function of the activity signal, pressure signal and/or the trigger threshold. For example, the window adjustment module 930 may temporarily shorten the window (i.e. reduces w) such as to allow the threshold to make a quick recovery from a brief isolated episode of increased noise, for example, due to a cough or a cannula bump that may be present in the activity signal. In one implementation, the window duration w may be a function of t_{L}, where t_{L}, (e.g., in seconds) is the time since the trigger threshold h exceeded its recent average (e.g., a recent average of calculated trigger threshold h values) by a significant margin, indicating a brief isolated episode of increased noise. In one implementation, the function is t_{L} minus some time (e.g., one), the recent average is a moving average (e.g., 5-second moving average), and the significant margin is 0.7 mmH₂O or other value in a range of, for example, about 0.4 or 0.10 mmH₂O.

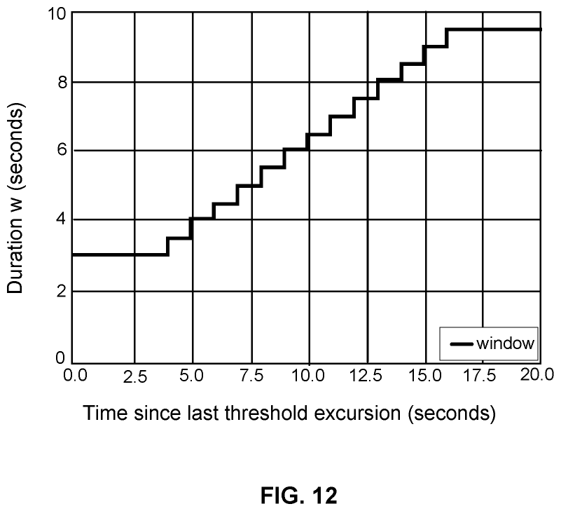

FIG. 12 is a graph illustrating another implementation of a function for window duration adjustment. In FIG. 12, the window duration w is plotted as a function of the time t_{L} (in seconds) since the trigger threshold excursion (i.e., the trigger threshold exceeded its recent average by a significant margin). The window duration gradually increases, such as with the function, from a lower limit (e.g., 3 seconds) immediately after the excursion to an upper limit (e.g., 10 seconds) after about 15 seconds. FIG. 12 shows the increase in the window duration as stepwise, but the increase could also be a smooth linear or nonlinear increase. Typically, such increases may repeatedly occur in the absence of an excursion that would otherwise reduce the duration.

In any case, the window adjustment module 930 maintains the window duration w between minimum and maximum limits, e.g. a lower limit of 3 seconds and an upper limit of 10 seconds.

The threshold update module 920 and the window adjustment module 930 may run at a low rate, e.g. updating the trigger threshold h and the window duration w at 2 Hz.

Optionally, the activity signal a(t) may be further processed to report activity metrics of the user, such as steps taken, and intensity and duration of exercise periods, and activity type.

In another implementation of the threshold module 720, the threshold h may be computed as a proportion of the recent average of peak values of the adjusted pressure signal Padj such as from values of a window W of the adjusted pressure signal Padj.

As mentioned above, the function of the trigger module 730 is to apply the trigger threshold h from the threshold module 720 to the adjusted pressure signal P_{adj} from the pressure module 710 to generate a bolus release control or trigger signal, such as a Boolean onset-of-inhalation signal trigger. Although in this example such a trigger signal may be Boolean, it is understood that the trigger signal may otherwise be any type of signal, such as a porportional control signal or other control signal, to operate (e.g., proportionally) or cause operation of any device, such as a control valve, for bolus release (such as for initiation and/or termination). Thus, generation of the control signal may be based on one or more comparisons of a pressure signal (e.g., adjusted pressure signal P_{adj}) and a variable theshold (e.g., trigger threshold h). In one implementation, trigger is asserted (i.e., a control signal that releases a bolus) when the following Boolean expression is true:
a) the adjusted pressure signal P_{adj} falls below the (negative) trigger threshold h continuously for at least a trigger confirmation period, equal in one implementation to 3 ms, OR has done so within a recent interval, e.g. 500 ms; AND
b) the time since the last assertion of trigger is greater than a blackout period, equal in one implementation to 1 second; AND
c) Expiration has been detected since the last assertion of trigger; AND
d) The elapsed time Ti since the start of the current inspiration is greater than a minimum inspiratory time value Timin.

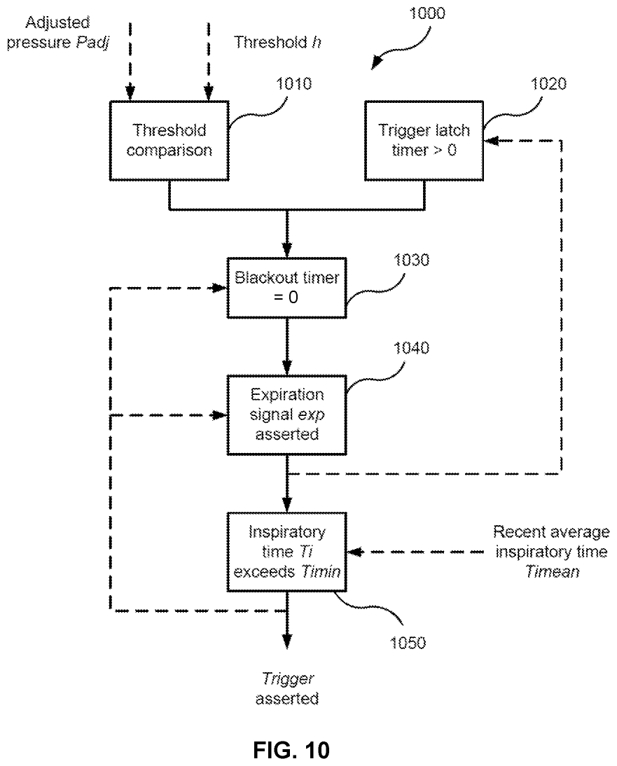

FIG. 10 is a schematic representation of an example process 1000 to achieve the above Boolean expression according to this implementation of the trigger module 730. Block 1010 performs a comparison such as to determine whether the adjusted pressure Padj falls below the threshold h continuously for at least a trigger confirmation period. This may represent the first part of condition (a) above. Increasing the trigger confirmation period increases the immunity of the trigger module 730 to noise but also increases the latency (delay) of inhalation onset detection. In one implementation, the trigger confirmation period may be adjusted within a range, e.g. 3 ms to 25 ms, based on recent inspiratory times, pressure signal-to-noise ratio, and/or the magnitude of recent pressure signal excursions. This may allow the trigger module 730 to avoid false triggering on noise while still triggering early enough for the bolus to be “effective.” (“Effective” may be understood to mean that the whole bolus will traverse the deadspace and arrive at the lung before exhalation starts. Thus, it might take part in gas exchange. As a guide, the bolus delivery should be complete by 60% of the inspiratory time to be effective.)

Block 1020 represents the second part of condition (a). A pending inspiration is equivalent to a “trigger latch” timer having a non-zero value. The trigger latch timer counts down the elapsed time since being restarted as described below. Block 1020 returns true if the trigger latch timer has not yet counted down to zero. In one implementation, the trigger latch timer is restarted with a value of 500 ms.

Block 1030 represents condition (b). Block 1030 checks the value of a “blackout timer” that counts down the elapsed time since being restarted by the previous assertion of trigger. Block 1030 returns true if the blackout timer has counted down to zero. In one implementation, the blackout timer is restarted with a value of one second. Block 1030 limits the bolus delivery rate rate to a maximum value (e.g. 60 per minute) equal to the reciprocal of the blackout timer restart value (a minimum time between boluses).

Block 1040 represents condition (c). Block 1040 checks whether the exp signal has been asserted by the monitoring module 740 (see below). The trigger module 730 clears the exp signal by the assertion of trigger by block 1050. Block 1040 ensures only one assertion of trigger occurs per inspiration.

Once block 1040 returns true, the trigger module 730 restarts the trigger latch timer (block 1020).

Block 1050 represents condition (d). Block 1050 delays the start of each bolus by the minimum value Timin to provide additional detection robustness by allowing greater time to confirm inhalation onset when time is available. Block 1050 may calculate the minimum value Timin as a function of one or more breathing and/or activity parameters, such as the recent average inspiratory time Timean and/or the maximum activity aₘₐₓ (equation(4)). The recent average inspiratory time Timean may be provided by the monitoring module 740 (see below).

In some implementations, the minimum value Timin may be reduced as the user's breathing rate rises, to a minimum of, for example, 0 ms. Such a reduction may be implemented when the time to deliver the bolus within the effective part of inspiration is minimal. The minimum value Timin may be increased such as to a maximum value as the breathing rate falls or maximum activity aₘₐₓ increases. Such a maximum value may be, for example in one implementation to 100 ms or a value between approximately 80 and 120 ms.

In one such implementation, Timin may be calculated by linear interpolation between a floor value dfloor and a ceiling value dceil, based on an interpolation parameter k:

Ti min=d_{floor}+k(d_{ceil}−d_{floor}) (5)

where k is linearly related to the maximum activity aₘₐₓ:

$$k = \frac{{a}_{max} - 200}{800} \qquad ( 6 )$$

That is, as the maximum activity amax increases, Timin increases towards the ceiling value dceil. The floor and ceiling values may be dependent on the recent average inspiratory time Timean:

$$\begin{matrix}{d}_{floor} = \frac{T i m e a n}{25} - 30 & ( 7 ) \\ {d}_{ceil} = \frac{3}{20} Timean & ( 8 )\end{matrix}$$

In other implementations, Timin may be randomly chosen in the interval [dfloor, dceil] or gradually increased over the interval [dfloor, dceil] to encourage entrainment of breathing rate toward a lower breathing rate. For these purposes, the derivation of dceil could be modified to allow greater delay given the breathing rate and therefore the duration Tpulse of the bolus, e.g.:

d_{ceil}=0.6Ti−Tpulse (9)

As mentioned above, the monitoring module 740 may also compute one or more breathing parameters of the user. In one implementation, the monitoring module estimates a breathing rate R of the user as the reciprocal of the recent breath duration. A breath duration is the length of the interval between successive instants when the signal trigger is asserted. The recent breath duration may be estimated as a moving average of the most recent breath durations. In one implementation, the three most recent breath durations are averaged to compute the recent breath duration. The breathing rate estimate R may be updated after each assertion of the signal trigger. If no breath is detected for 7.5 seconds—corresponding to 8 BPM—the recent breath duration is reset to a default value of 3 seconds—corresponding to 20 BPM. This ensures in cases of lost signal the dependent modules default to a typical breathing rate and not an extremely low breathing rate.

The monitoring module 740 may also estimate the user's inspiratory time T_{I}. This may be done by analysing the adjusted pressure signal P_{adj} from the pressure module 710. In one implementation, the inspiratory time T_{I} is the duration for which the adjusted pressure P_{adj} has continuously remained below zero. In another implementation, upon assertion of the signal trigger, the monitoring module 740 evaluates the recent history of P_{adj} to determine the actual onset of inhalation. Once the adjusted pressure P_{adj} exceeds zero for a minimum duration, e.g. 250 ms, indicating exhalation has started, the monitoring module 740 evaluates the recent history of P_{adj} to determine the actual onset of exhalation. The inspiratory time T_{I} is the length of the interval between the onset of inhalation and the onset of exhalation.

The monitoring module 740 may also average several recent values of the inspiratory time T_{I} to calculate the recent average inspiratory time Timean.

The monitoring module 740 may also detect expiration. In one implementation, the monitoring module 740 asserts a Boolean signal exp indicating expiration if the adjusted pressure signal P_{adj} remains above an expiratory threshold for a minimum expiration period, equal in one implementation to 250 ms. In another implementation, the monitoring module 740 determines an estimate of respiratory phase according to the time since the previous assertion of trigger as a proportion of the recent breath duration (e.g., time_since_last_trigger/recent_breath_duration). When the determined estimate of respiratory phase is less than 0.5, the expiratory threshold applied is the minimum threshold hmin. When the determined estimate of respiratory phase is greater than 0.5, the expiratory threshold is the negative of the minimum threshold hmin. This may help to ensure that both nose and mouth expiration are detected while reducing the occurrence of false detections in the case of severe flow limitation.

### General Remarks

In the present disclosure, certain U.S. patents, U.S. patent applications, and other materials (e.g., articles) have been incorporated by reference. The text of such U.S. patents, U.S. patent applications, and other materials is, however, only incorporated by reference to the extent that no conflict exists between such text and the other statements and drawings set forth herein. In the event of such conflict, then any such conflicting text in such incorporated by reference U.S. patents, U.S. patent applications, and other materials is specifically not incorporated by reference in this patent.

Further modifications and alternative embodiments of various aspects of the present technology may be apparent to those skilled in the art in view of this description. Accordingly, this description is to be construed as illustrative only and is for the purpose of teaching those skilled in the art the general manner of carrying out the technology. It is to be understood that the forms of the technology shown and described herein are to be taken as embodiments. Elements and materials may be substituted for those illustrated and described herein, parts and processes may be reversed, and certain features of the technology may be utilized independently, all as would be apparent to one skilled in the art after having the benefit of this description of the technology. Changes may be made in the elements described herein without departing from the spirit and scope of the technology as described in the appended claims.

### LABEL LIST

| | |
|---|---|
| oxygen concentrator | 100 |
| inlet | 101 |
| inlet | 105 |
| accumulator | 106 |
| inlet muffler | 108 |
| inlet valves | 122 |
| inlet valves | 124 |
| concentrator outlet | 130 |
| valves | 132 |
| muffler | 133 |
| outlet valves | 134 |
| check valves | 142 |
| check valves | 144 |
| flow restrictors | 151 |
| valve | 152 |
| flow restrictors | 153 |
| valves | 154 |
| flow restrictors | 155 |
| supply valve | 160 |
| chamber | 162 |
| oxygen sensor | 165 |
| ultrasonic emitter | 166 |
| receiver | 168 |
| outer housing | 170 |
| fan | 172 |
| outlet | 173 |
| outlet port | 174 |
| small orifice flow restrictor | 175 |
| power supply | 180 |
| flow rate sensor | 185 |
| filter | 187 |
| connector | 190 |
| conduit | 192 |
| pressure sensor | 194 |
| airway delivery device | 196 |
| mouthpiece | 198 |
| compression system | 200 |
| compressor | 210 |
| compressor outlet | 212 |
| motor | 220 |
| external armature | 230 |
| air transfer device | 240 |
| compressor outlet conduit | 250 |
| canister system | 300 |
| canister | 302 |
| canister | 304 |
| base | 315 |
| outlet | 325 |
| gases | 327 |
| controller | 400 |
| processors | 410 |
| memory | 420 |
| control panel | 600 |
| input port | 605 |
| power button | 610 |
| button | 620 |
| buttons | 622 |
| button | 624 |
| button | 626 |
| button | 630 |
| button | 635 |
| button | 640 |
| button | 650 |
| LED | 655 |
| adaptive triggering system | 700 |
| pressure module | 710 |
| threshold module | 720 |
| trigger module | 730 |
| monitoring module | 740 |
| pressure module | 800 |
| noise reduction filter | 810 |
| offset calculation module | 820 |
| temperature compensation module | 830 |
| noise reduction filter | 840 |
| bolus removal module | 850 |
| threshold module | 900 |
| activity estimation filter | 910 |
| threshold update module | 920 |
| window adjustment module | 930 |
| schematic representation | 1000 |
| block | 1010 |
| block | 1020 |
| block | 1030 |
| block | 1040 |
| block | 1050 |

What is claimed is:

1. A method of generating a trigger signal for controlling release of a bolus of oxygen enriched gas from an oxygen concentrator, the method comprising:
calculating a trigger threshold from a pressure signal representing an airway pressure of a user,
comparing the pressure signal with the trigger threshold, and
generating, based on the comparison, the trigger signal for controlling release of the bolus.

2. The method of claim 1, wherein calculating the trigger threshold comprises computing an activity signal from the pressure signal.

3. The method of claim 2 wherein the activity signal represents activity other than respiration activity.

4. The method of any one of claims 2 to 3, wherein calculating the trigger threshold comprises decreasing a sensitivity of the trigger threshold with an increase in an indication of activity in the activity signal.

5. The method of claim 4, wherein decreasing the sensitivity of the trigger threshold comprises making the trigger threshold more negative.

6. The method of any of claims 2 to 5, wherein calculating the trigger threshold comprises increasing a sensitivity of the trigger threshold with a decrease in an indication of activity in the activity signal.

7. The method of claim 6, wherein increasing the sensitivity of the trigger threshold comprises making the trigger threshold less negative.

8. The method of any one of claims 4 to 7 wherein the indication of activity is derived as a function of a window of values of the activity signal.

9. The method of claim 8 wherein a duration of the window varies as a function of time since the trigger threshold exceeded an average of trigger threshold values.

10. The method of claim 9 wherein the function of time is configured to shorten the duration of the window.

11. The method of any one of claims 8 and 9 wherein the function of time is further configured to gradually increase the duration of the window to a limit.

12. The method of any of claims 2 to 11, wherein calculating the trigger threshold comprises setting the trigger threshold according to a function of (a) a scaling constant, and (b) a maximum value of a window of values of the activity signal.

13. The method of claim 12 wherein the function comprises multiplying the scaling constant and the maximum value and reversing a sign of a value of the function.

14. The method of any one of claims 12 to 13, further comprising varying the scaling constant with the maximum value.

15. The method of any one of claims 12 to 13, further comprising varying the scaling constant with a breathing rate of the user.

16. The method of any of claims 2 to 15, wherein computing the activity signal comprises high pass filtering the pressure signal.

17. The method of any of claims 1 to 16, wherein comparing the pressure signal with the trigger threshold comprises determining whether the pressure signal falls below the trigger threshold continuously for at least a trigger confirmation period.

18. The method of claim 17, wherein generating the trigger signal comprises asserting a Boolean trigger signal.

19. The method of claim 18, further comprising detecting an expiration of the user.

20. The method of claim 19, wherein assertion of the Boolean trigger signal is conditioned on a detection of an expiration since the last assertion of the Boolean trigger signal.

21. The method of any of claims 19 to 20, wherein detecting an expiration comprises determining whether the pressure signal remains above an expiratory threshold for a minimum expiration period.

22. The method of any of claims 18 to 21, wherein assertion of the Boolean trigger signal is conditioned on a time since the last assertion of the Boolean trigger signal exceeding a minimum time between boluses.

23. The method of any of claims 18 to 22, wherein assertion of the Boolean trigger signal is conditioned on a duration of a current inspiration being greater than a minimum inspiratory time.

24. The method of claim 23, further comprising calculating the minimum inspiratory time as a function of a recent average inspiratory time.

25. The method of claim 24, wherein calculating the minimum inspiratory time comprises choosing a value between a floor value and a ceiling value, wherein at least one of the floor value and the ceiling value increases with the recent average inspiratory time.

26. The method of any of claims 1 to 25, wherein the pressure signal is an adjusted pressure signal.

27. The method of claim 26, further comprising generating the adjusted pressure signal by computing values for the adjusted pressure signal that adjust at least one period of a measured pressure signal that coincides with a bolus release to remove an effect of the bolus release on the measured pressure signal.

28. The method of claim 27, wherein computing values for the adjusted pressure signal comprises interpolating between a last measured pressure value prior to the bolus release and a first measured pressure value after the bolus release.

29. The method of claim 28 wherein computing values for the adjusted pressure signal further comprises interpolating values for a settling period of the adjusted pressure signal that occurs after the first measured pressure value.

30. The method of any of claims 26 to 29, wherein the adjusted pressure signal is generated by filtering so as to achieve one or both of: (a) removal of very short duration, large magnitude impulses, and (b) removal of periodic device noise.

31. The method of any of claims 26 to 30, wherein the adjusted pressure signal is generated by compensating for a temperature of the oxygen concentrator.

32. The method of claim 31, wherein compensating for the temperature comprises computing a pressure offset from a signal representing the temperature of the oxygen concentrator.

33. The method of any of claims 1 to 32, further comprising estimating a breathing rate of the user from successive instants of bolus release.

34. The method of any of claims 1 to 33, further comprising estimating an inspiratory time of the user.

35. A computer-readable medium having encoded thereon computer-readable instructions that when executed by a controller of an oxygen concentrator cause the controller to perform the method of any one of claims 1 to 34.

36. A portable oxygen concentrator comprising:
an outlet, the outlet suitable for pneumatic coupling with a delivery device, the delivery device for delivering, in use, oxygen enriched gas to a user;
at least two canisters including gas separation adsorbent, the gas separation adsorbent configured for gas separation of at least some nitrogen from air in the at least two canisters to produce the oxygen enriched gas;
a compression system comprising a compressor coupled to at least one of the canisters to compress air during operation to promote the gas separation;
an accumulator coupled to one or more of the canisters, to accumulate the oxygen enriched gas produced in one or more of the canisters during use, the accumulator pneumatically coupled to the outlet;
one or more sensors; and
a controller, including one or more processors, and a set of valves coupled to the controller, the controller configured to control operation of the set of valves to (a) produce the oxygen enriched gas into the accumulator and (b) release the produced oxygen enriched gas from the accumulator in at least one bolus, the controller further configured to operate with the computer-readable medium of claim 35.

37. An adaptive triggering system for an oxygen concentrator, the system comprising:
a threshold module configured to repeatedly calculate a trigger threshold from a pressure signal representing an airway pressure of a user;
a trigger module configured to:
compare the pressure signal with the trigger threshold; and
generate, based on the comparison, a trigger signal to control release of a bolus.

38. The adaptive triggering system of claim 37, wherein the pressure signal is an adjusted pressure signal, and wherein the system further comprises a pressure module configured to generate the adjusted pressure signal by adjusting at least one period of a measured pressure signal that coincides with a bolus release to remove an effect of the bolus release on the measured pressure signal.

39. The adaptive triggering system of claim 38, further comprising a temperature sensor configured to generate a temperature signal, wherein the pressure module is configured to generate the adjusted pressure signal using the temperature signal to compensate for temperature of the oxygen concentrator.

40. The adaptive triggering system of any of claims 37 to 39, further comprising a monitoring module configured to calculate one or more breathing parameters of the user from successive instants of bolus release.

41. An oxygen concentrator comprising the adaptive triggering system of any one of claims 37 to 40.

42. An adaptive triggering system for an oxygen concentrator, the system comprising:
means for repeatedly calculating a trigger threshold from a pressure signal representing an airway pressure of a user,
means for comparing the pressure signal with the trigger threshold, and
means for generating a trigger signal to control release of a bolus of oxygen based on the comparison.
